(19) United States
(12) Patent Application Publication
(10) Pub. No.: US 2006/0013789 A1
(43) Pub. Date: Jan. 19, 2006

(54) Cosmetic composition with improved staying power

(75) Inventors: Xavier Blin, Paris (FR); Momoko Shimizu, Kawasaki-shi (JP)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/181,948

(22) Filed: Jul. 15, 2005

Related U.S. Application Data
(60) Provisional application No. 60/591,928, filed on Jul. 29, 2004.

(30) Foreign Application Priority Data
Jul. 16, 2004 (FR) 04 51558

(51) Int. Cl. A61K 8/894

(52) U.S. Cl. 424/70.12

(57) ABSTRACT

Cosmetic makeup and/or care compositions for application to the skin, lips and/or epidermal derivatives, may include silicone polymers and hydrocarbon ester oils with low molecular weight.

### CROSS-REFERENCE TO RELATED APPLICATIONS

This non-provisional application claims the benefit of French Application No. 04 51558 filed on Jul. 16, 2004 and U.S. Provisional Application No. 60/591,928 filed on Jul. 29, 2004, the disclosures of which are incorporated herein by reference in their entirety.

### BACKGROUND

Generally speaking, the purpose of cosmetic compositions is to impart an aesthetic effect at the time of application and to maintain this aesthetic effect over time. Cosmetic compositions are required, in particular, to withstand the various external factors that are liable to alter their aesthetic effect, such as perspiration or tears, in the case of a foundation, or saliva, in the case of a lipstick.

Cosmetic compositions, such as lipsticks, for example, must not migrate into fine lines or wrinkles, or undergo transfer to a fabric. Cosmetic compositions must also be pleasant to apply and must maintain a sensation of comfort over time, while retaining satisfactory aesthetic properties.

Japanese patent applications JP 6-305933 and JP 7-330547 describe compositions comprising silicone alkylglyceryl ether derivatives. Compositions having an oily base and comprising polyglycerylated silicone derivatives or fluoroalkylpolyglycerylated silicone derivatives have been proposed in JP 6-157236, JP 9-071504 and JP 10-310504. Compositions comprising silicone alkylglycerol derivatives have also been described in EP 0 475 130 and in JP 2-844453 and JP 2-587797. Other compositions comprising silicone derivatives hydroxylated with saccharides, with butylene glycol or with glycerol have been described in JP 5-186596 and JP 6-145023.

### SUMMARY

However, in the presence of water, these compositions form a gel on the skin's surface, leading to a sensation of discomfort over time.

However, there presently is no completely satisfactory cosmetic composition that has desired aesthetic characteristics and that can provide satisfactory color permanence, gloss and comfort.

Exemplary embodiments provide cosmetic makeup and/or care compositions comprising a particular silicone polymer and a hydrocarbon ester oil with a low molecular weight. Such cosmetic compositions can exhibit improved color fastness while having a satisfactory or improved gloss and comfort.

Exemplary embodiments of cosmetic compositions disclosed herein are, more particularly, makeup and/or care products for application to the skin, lips and/or epidermal derivatives. These compositions may be lipsticks; lip balms; lip pencils; liquid foundations or solid foundations, such as those cast as a stick or in a dish; concealer products and skin colouring products; temporary tattoos; eye makeup products such as eyeliners, such as those in the form of pencils, and mascaras, such as those in the form of cakes, and eye shadows.

Exemplary embodiments provide cosmetic compositions include, in a physiologically acceptable medium, hydrocarbon esters comprising less than 40 carbon atoms and silicone polymers of general formula (I), R¹ₐR²_{b}R³_{c}SiO_{(4-a-b-c)/2}, in which: a varies is in a range of from about 1 to about 2.5 and b and c independently of one another vary from 0.001 to 1.5, b and c each are independently in a range of from about 0.001 to about 1.5, R¹, which is may be identical or different at each occurrence, is selected chosen from: C₁ to C₃₀ alkyl radicals, substituted where appropriate by one or more fluorine atoms, amino and/or carboxyl groups, C₁ to C₃₀ alkyl radicals, substituted by one or more fluorine atoms, amino and/or carboxyl groups, aryl and aralkyl radicals, radicals of general formula (II), —C_{d}H_{2d}—O—(C₂H₄O)ₑ(C₃H₆O)_{f}R⁴, wherein: R⁴ is chosen from C₁ to C₃₀ hydrocarbon radicals and or a radicals R⁵—(CO)—, with in which R⁵ is chosen from C₁ to C₃₀ hydrocarbon radicals, and d, e and f being integers such that d varies is an integer in a range of from 0 to about 15 and e and f, independently of one another, vary from 0 to 50, and e and f are each integers that are independently in a range of from 0 to about 50, combinations thereof, R2 is a radical represented by the general formula (III), -Q-O—X, wherein: Q is chosen from divalent C₂ to C₂₀ hydrocarbon radicals that which can include at least one ether bond and/or at least one ester bond, and X being is chosen from a polyhydroxylated hydrocarbon radicals, wherein with the proviso that, when R² is a group radical of general formula (IIIA), —C₃H₆—O[CH₂CH(OH)CH₂O]ₙH, in which n is an integer varying in a range of from 1 to 5, R¹ is not a C₁₋₂ alkyl radical; R³ is an organosiloxane group of general formula (IV): 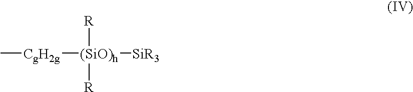
, wherein: the radicals R are each radicals independently chosen from the group consisting of representing, independently of one another, a radical selected from C₁ to C₃₀ alkyl radicals, C₁ to C₃₀ alkyl radicals substituted where appropriate with one or more fluorine atoms, and aryl and aralkyl radicals, g and h being integers such that g is an integer in a range of from 1 to about 5, and h varies is an integer in a range of from 0 to about 500, and containing less than about 15% by weight of volatile oil relative to the total weight of the composition.

Exemplary embodiments provide cosmetic compositions comprising, in a physiologically acceptable medium, at least one hydrocarbon monoester comprising less than 40 carbon atoms and at least one silicone polymer of general formula (I), R¹ₐR²_{b}R³_{c}SiO_{(4-a-b-c)/2}, in which: a is in a range of from about 1 to about 2.5, b and c are each independently in a range of from about 0.001 to about 1.5, R¹, which may be identical or different at each occurrence, is a radical chosen from the group consisting of: C¹, to C₃₀ alkyl radicals, C₁ to C₃₀ alkyl radicals substituted with one or more fluorine atoms, amino and/or carboxyl groups, aryl and aralkyl radicals, radicals of general formula (II), —C_{d}H_{2d}—O—(C₂H₄O)ₑ(C₃H₆O)_{f}R⁴, wherein: R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals, d is an integer in a range of from 0 to about 15, and e and f are each independently integers in a range of from 0 to about 50, and combinations thereof, R² is a radical represented by the general formula (III), -Q-O—X, wherein: Q is a divalent C₂ to C₂₀ hydrocarbon radical that can include at least one ether bond and/or at least one ester bond, and X is a polyhydroxylated hydrocarbon radical, wherein when R² is a radical of general formula (IIIA), —C₃H₆—O[CH₂CH(OH)CH₂O]ₙH, in which n is an integer in a range of from 1 to 5, R¹ is not a C₁₋₂ alkyl radical; R³ is an organosiloxane of general formula (IV): 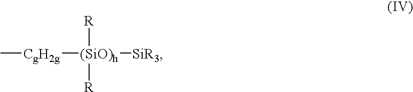
wherein: the radicals R are each radicals independently chosen from the group consisting of C₁ to C₃₀ alkyl radicals, C₁ to C₃₀ alkyl radicals substituted with one or more fluorine atoms, and aryl and aralkyl radicals, g is an integer in a range of from 1 to about 5, and h is an integer in a range of from 0 to about 500.

Exemplary embodiments of the present invention provide anhydrous cosmetic compositions comprising, in a physiologically acceptable medium, hydrocarbon esters comprising less than 40 carbon atoms and silicone polymers of general formula (I), R¹ₐR²_{b}R³_{c}SiO_{(4-a-b-c)/2}, in which: a is in a range of from about 1 to about 2.5, b and c are each independently in a range of from about 0.001 to about 1.5, R¹, which may be identical or different at each occurrence, is a radical chosen from the group consisting of: C₁ to C₃₀ alkyl radicals, C₁ to C₃₀ alkyl radicals substituted with one or more fluorine atoms, amino and/or carboxyl groups, aryl and aralkyl radicals, radicals of general formula (II), —C_{d}H_{2d}—O—(C₂H₄O)ₑ(C₃H₆O)_{f}R⁴, wherein: R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals, d is an integer in a range of from 0 to about 15, and e and f are each independently integers in a range of from 0 to about 50, and combinations thereof, R² is a radical of general formula (III), -Q-O—X, wherein: Q is a divalent C₂ to C₂₀ hydrocarbon radical that can include at least one ether bond and/or at least one ester bond, and X is a polyhydroxylated hydrocarbon radical, wherein when R² is a radical of general formula (IIIA), —C₃H₆—O[CH₂CH(OH)CH₂O]ₙH, in which n is an integer in a range of from 1 to about 5, R³ is not a C₁₋₂ alkyl radical; R³ is an organosiloxane of general formula (IV), 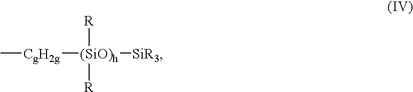
wherein: the radicals R are each radicals independently chosen from the group consisting of C₁ to C₃₀ alkyl radicals, C₁ to C₃₀ alkyl radicals substituted with one or more fluorine atoms, and aryl and aralkyl radicals, g is an integer in a range of from 1 to about 5, and h is an integer in a range of from 0 to about 500.

Exemplary embodiments of the present invention provide anhydrous cosmetic compositions comprising, in a physiologically acceptable medium, fluid silicon compounds and hydrocarbon esters comprising less than 40 carbon atoms and/or volatile oils, and silicone polymers of general formula (I), R¹ₐR²_{b}R³_{c}SiO_{(4-a-b-c)/2}, in which: a is in a range of from about 1 to about 2.5, b and c are each independently in a range of from about 0.001 to about 1.5, R¹, which may be identical or different at each occurrence, is a radical chosen from the group consisting of: C₁ to C₃₀ alkyl radicals, C₁ to C₃₀ alkyl radicals substituted with one or more fluorine atoms, amino and/or carboxyl groups, aryl and aralkyl radicals, radicals of general formula (II), —C_{d}H_{2d}—O—(C₂H₄O)ₑ(C₃H₆O)_{f}R⁴ wherein: R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals, d is an integer in a range of from 0 to about 15, and e and f are each independently integers in a range of from 0 to about 50, and combinations thereof, R² is a radical of general formula (III), -Q-O—X, wherein: Q is a divalent C₂ to C₂₀ hydrocarbon radical that can include at least one ether bond and/or at least one ester bond, and X is a polyhydroxylated hydrocarbon radical, R³ is an organosiloxane of general formula (IV), 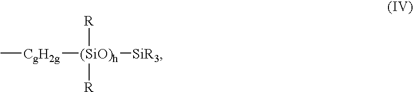
wherein: the radicals R are each radicals independently chosen from the group consisting of C₁ to C₃₀ alkyl radicals, C₁ to C₃₀ alkyl radicals substituted with one or more fluorine atoms, and aryl and aralkyl radicals, g is an integer in a range of from 1 to about 5, and h is an integer in a range of from 0 to about 500.

Exemplary embodiments of the present invention provide anhydrous cosmetic compositions comprising, in a physiologically acceptable medium, hydrocarbon esters containing less than 40 carbon atoms, volatile oils and silicone polymers of general formula (I), R¹ₐR²_{b}R³_{c}SiO_{(4-a-b-c)/2} in which: a is in a range of from about 1 to about 2.5, b and c are each independently in a range of from about 0.001 to about 1.5, R¹, which may be identical or different at each occurrence, is a radical chosen from the group consisting of: C₁ to C₃₀ alkyl radicals, C₁ to C₃₀ alkyl radicals substituted with one or more fluorine atoms, amino and/or carboxyl groups, aryl and aralkyl radicals, radicals of general formula (II), —C_{d}H_{2d}—O—(C₂H₄O)ₑ(C₃H₆O)_{f}R⁴, wherein: R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals, d is an integer in a range of from 0 to about 15, and e and f are each independently integers in a range of from 0 to about 50, and combinations thereof, R² being a radical of general formula (II), -Q-O—X, wherein: Q is a divalent C₂ to C₂₀ hydrocarbon radical that can include at least one ether bond and/or at least one ester bond, and X is a polyhydroxylated hydrocarbon radical, R³ being an organosiloxane of general formula (IV), 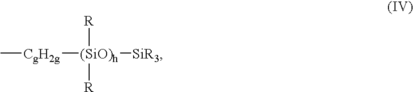
wherein: the radicals R are each radicals independently chosen from the group consisting of C₁ to C₃₀ alkyl radicals, C₁ to C₃₀ alkyl radicals substituted with one or more fluorine atoms, and aryl and aralkyl radicals, g is an integer in a range of from 1 to about 5, and h is an integer in a range of from 0 to about 500.

Exemplary embodiments of the present invention provide anhydrous cosmetic compositions comprising, in a physiologically acceptable medium, at least two esters containing less than 40 carbon atoms and silicone polymers of general formula (I), R¹ₐR²_{b}R³_{c}SiO_{(4-a-b-c)/2}, in which: a is in a range of from about 1 to about 2.5, b and c are each independently in a range of from about 0.001 to about 1.5, R¹, which may be identical or different at each occurrence, is a radical chosen from the group consisting of: C₁ to C₃₀ alkyl radicals, C₁ to C₃₀ alkyl radicals substituted with one or more fluorine atoms, amino and/or carboxyl groups, aryl and aralkyl radicals, radicals of general formula (II), —C_{d}H_{2d}—O—(C₂H₄O)ₑ(C₃H₆O)_{f}R⁴, wherein: R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals, d is an integer in a range of from 0 to about 15, and e and f are each independently integers in a range of from 0 to about 50, and combinations thereof, R² being a radical of general formula (III), -Q-O—X, wherein: Q is a divalent C₂ to C₂₀ hydrocarbon radical that can include at least one ether bond and/or at least one ester bond, and X is a polyhydroxylated hydrocarbon radical, R³ being an organosiloxane of general formula (IV): 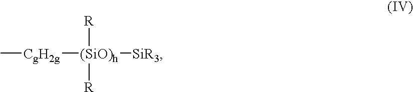
wherein: the radicals R are each radicals independently chosen from the group consisting of C₁ to C₃₀ alkyl radicals, C₁ to C₃₀ alkyl radicals substituted with one or more fluorine atoms, and aryl and aralkyl radicals, g is an integer in a range of from 1 to about 5, and h is an integer in a range of from 0 to about 500.

Exemplary embodiments of the present invention provide anhydrous cosmetic compositions comprising, in a physiologically acceptable medium, hydrocarbon esters containing fewer than 22 carbon atoms and silicone polymers of general formula (I), R¹ₐR²_{b}R³_{c}SiO_{(4-a-b-c)/2}, in which: a is in a range of from about 1 to about 2.5, b and c are each independently in a range of from about 0.001 to about 1.5, R¹, which may be identical or different at each occurrence, is a radical chosen from the group consisting of: C₁ to C₃₀ alkyl radicals, C₁ to C₃₀ alkyl radicals substituted with one or more fluorine atoms, amino and/or carboxyl groups, aryl and aralkyl radicals, and radicals of general formula (II), —C_{d}H_{2d}—O—(C₂H₄O)ₑ(C₃H₆O)_{f}R⁴, wherein: R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals, d is an integer in a range of from 0 to about 15, and e and f are each independently integers in a range of from 0 to about 50, and combinations thereof, R² being a radical of general formula (III), -Q-O—X, wherein: Q is a divalent C₂ to C₂₀ hydrocarbon radical that can include at least one ether bond and/or at least one ester bond, and X is a polyhydroxylated hydrocarbon radical, R³ being an organosiloxane of general formula (IV): 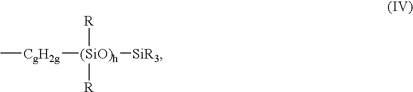
wherein: the radicals R are each radicals independently chosen from the group consisting of C₁ to C₃₀ alkyl radicals, C₁ to C₃₀ alkyl radicals substituted with one or more fluorine atoms, and aryl and aralkyl radicals, g is an integer in a range of from 1 to about 5, and h is an integer in a range of from 0 to about 500.

Exemplary embodiments of the present invention provide methods of preparing anhydrous cosmetic compositions comprising: combining hydrocarbon esters comprising less than 40 carbon atoms, silicone compounds and/or volatile oils different from the silicone polymers of general formula (I), R¹ₐR²_{b}R³_{c}SiO_{(4-a-b-c)/2}, in which: a is in a range of from about 1 to about 2.5, b and c are each independently in a range of from about 0.001 to about 1.5, R¹, which may be identical or different at each occurrence, is a radical chosen from the group consisting of: C₁ to C₃₀ alkyl radicals, C₁ to C₃₀ alkyl radicals substituted with one or more fluorine atoms, amino and/or carboxyl groups, aryl and aralkyl radicals, radicals of general formula (II), —C_{d}H_{2d}—O—(C₂H₄₀)ₑ(C₃H₆₀)_{f}R⁴, wherein: R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals, d is an integer in a range of from 0 to about 15, and e and f are each independently integers in a range of from 0 to about 50, and combinations thereof, R² is a radical of general formula (III), -Q-O—X, wherein: Q is a divalent C₂ to C₂₀ hydrocarbon radical that can include at least one ether bond and/or at least one ester bond, and X is a polyhydroxylated hydrocarbon radical, R³ is an organosiloxane of general formula (IV): 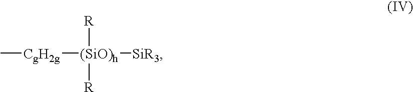
wherein: the radicals R are each radicals independently chosen from the group consisting of C₁ to C₃₀ alkyl radicals, C₁ to C₃₀ alkyl radicals substituted with one or more fluorine atoms, and aryl and aralkyl radicals, g is an integer in a range of from 1 to about 5, and h is an integer in a range of from 0 to about 500.

### DETAILED DESCRIPTION OF EMBODIMENTS

Exemplary embodiments overcome the drawbacks of the above-discussed cosmetic compositions and provide cosmetic compositions that exhibit improved staying power on keratin materials, without detrimental effects on gloss characteristics. In embodiments, exemplary cosmetic compositions exhibit improved gloss characteristics and/or improved comfort.

As used herein, the term “keratin materials” is includes, but is not limited to, the skin, mucosae, such as the lips, the nails and the keratin fibres, as exemplified by the eyelashes and the hair.

Exemplary cosmetic compositions may be applied on the skin and lips.

Use of a silicone polymer of general formula (I):
R¹ₐR²_{b}R³_{c}SiO_{(4-a-b-c)/2} (I)
in which R¹ is an alkyl radical, R² is represented by the general formula (III):
   Q-O—X (III)
in which Q is a divalent hydrocarbon radical and X is a polyhydroxylated hydrocarbon radical, and R³ is an organosiloxane group of general formula (IV): 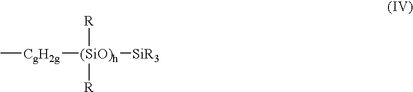
in which the radicals R are each independently an alkyl radical, in combination with at least one film former provides cosmetic compositions that have improved staying power without detriment to the gloss. In some embodiments, such cosmetic compositions can exhibit improved gloss and/or comfort.

In exemplary embodiments, anhydrous cosmetic compositions including in a physiologically acceptable medium at least one fluid silicone compound, at least one hydrocarbon ester comprising less than 40 carbon atoms and/or at least one volatile oil, and at least one silicone polymer of general formula (I):
R¹ₐR²_{b}R³_{c}SiO_{(4-a-b-c)/2} (I)
in which:
   a is an integer in the range of from 1 to 2.5, and b and c are each independently in the range of from 0.001 to 1.5.
   In general formula (I), R¹, which is may be the same or different at each occurrence of R¹, is chosen from substituted and unsubstituted C₁ to C₃₀ alkyl radicals in which the substituents may be one or more fluorine atoms, amino and/or carboxyl groups, aryl and aralkyl radicals, and radicals of general formula (II):
      —C_{d}H_{2d}—O—(C₂H₄O)ₑ(C₃H₆O)_{f}R⁴ (II).

In general formula (II), R⁴ is a radical that is chosen from C₁ to C₃₀ hydrocarbon radicals and R⁵—(CO)— radicals in which R⁵ is a C₁ to C₃₀ hydrocarbon radical, and d is an integer in the range of from 0 to 15 and e and f are independently chosen integers in the range of from 0 to 50, and combinations thereof.

In general formula (I), R2 is a radical represented by the general formula
-Q-O—X (III)
in which Q is a divalent C₂ to C₂₀ hydrocarbon radical that may include at least one ether bond and/or at least one ester bond, and X is a polyhydroxylated hydrocarbon radical.

R³, in general formula (I), is an organosiloxane of general formula (IV): 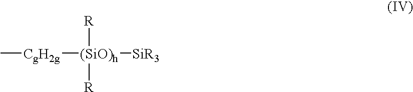
in which each is independently chosen from substituted and unsubstituted C₁ to C₃₀ alkyl radicals, in which the substituents may be one or more fluorine atoms, and aryl and aralkyl radicals, g is an integer in the range of from 1 to 5 and h is an integer in the range of from 0 to 500.

In additional exemplary embodiments, anhydrous cosmetic compositions include, in a physiologically acceptable medium, at least one hydrocarbon ester comprising less than 40 carbon atoms, at least one volatile oil and at least one silicone polymer of general formula (I), as defined above.

Additional exemplary embodiments provide cosmetic lip makeup compositions that include, in a physiologically acceptable medium, at least two esters comprising less than 40 carbon atoms and at least one silicone polymer of general formula (I), as defined above.

Additional exemplary embodiments provide cosmetic compositions that include, in a physiologically acceptable medium, at least one hydrocarbon ester containing less than 22 carbon atoms and at least one silicone polymer of general formula (I), as defined above.

In additional exemplary embodiments, anhydrous cosmetic compositions include, in a physiologically acceptable medium, at least one hydrocarbon ester comprising less than 40 carbon atoms and at least one silicone polymer of general formula (I), as defined above, in which, when R2 is a group of general formula (IIIA):
—C₃H₆—O[CH₂CH(OH)CH₂O]ₙH (IIIA)
in which n is an integer in the range of from 1 to 5, R¹ is not a C₁₋₂ alkyl radical, and containing less than 15% by weight of volatile oil relative to the total weight of the composition.

Further exemplary embodiments provide cosmetic compositions including, in a physiologically acceptable medium, at least one hydrocarbon monoester comprising less than 40 carbon atoms and at least one silicone polymer of general formula (I), as defined above, with the proviso that, when R² is a group of general formula (IIIA):
—C₃H₆—O[CH₂CH(OH)CH₂O]ₙH (IIIA)
in which n is an integer in the range of from 1 to 5, R¹ is not a C₁₋₂ alkyl radical.

Additional exemplary embodiments provide anhydrous cosmetic compositions including, in a physiologically acceptable medium, at least one ester comprising less than 40 carbon atoms and at least one silicone polymer of general formula (I), as defined above, in which, when R² is a group of general formula (IIIA):
—C₃H₆—O[CH₂CH(OH)CH₂O]ₙH (IIIA)
in which n is an integer in the range of from 1 to 5, R¹ is not a C₁₋₂ alkyl radical.

Further exemplary embodiments provide cosmetic compositions for making up and/or caring for the lips and/or the skin, and provide a lipstick.

Exemplary embodiments may also provide a synthetic support on which is present, over some or all of its surface, at least one layer of a cosmetic composition according to embodiments.

Further exemplary embodiments provide methods of making up and/or caring for keratin materials, such as the skin and/or lips, which include applying to the keratin materials at least one cosmetic composition in accordance with embodiments.

As used herein, “improved staying power” encompasses improved water resistance and/or oil resistance and/or reduced transfer.

The cosmetic compositions according to exemplary embodiments of the invention may be in the form of a paste, liquid, gel, cream or solid. In particular embodiments, cosmetic compositions are in cast form, such as in the form of a stick. Cosmetic compositions of exemplary embodiments may also be in the form of simple oil-in-water or water-in-oil emulsions or multiple emulsions, or in the form of anhydrous, solid or flexible gels.

In particular embodiments, cosmetic compositions are in an anhydrous form.

The term “composition in cast form,” as used herein, encompasses solid or semi-solid compositions obtained after cooling compositions that have been introduced into a mold while in a liquid or melt state. These exemplary cosmetic compositions may be cast in the form of a stick or crayon, or in a dish.

In particular exemplary embodiments, cosmetic compositions according to the invention are in cast form, i.e., in solid or semi-solid form, and may be in the form of a stick.

To determine the hardness of a cosmetic composition in accordance with exemplary embodiments, a stick of the composition having a circular section 12.7 mm in diameter may be prepared. The stick may be cast and then kept at a temperature of 20° C. for 24 hours before measurement.

The hardness may be measured by the “cheese wire” method, which consists of cutting the stick transversely by means of a rigid tungsten wire 250 μm in diameter, by advancing the wire relative to the stick at a speed of 100 mm/min. The hardness corresponds to the maximum shearing force exerted by the wire on the stick at 20° C., this force being measured by means of a DFGS2 dynamometer sold by Indelco-Chatillon. The hardness is expressed in grams.

According to this method, the hardness of cosmetic compositions of exemplary embodiments in accordance with the invention that are in stick form may vary from about 50 to about 300 g, such as from about 70 to about 250 g or from about 100 to about 230 g.

Exemplary embodiments of cosmetic compositions according to the present invention may exhibit improved colour permanence, as manifested, for example, by reduced migration or reduced transfer of colour, improved colour fastness to water improved colour fastness to oil, and/or reduced migration during application of the cosmetic compositions.

Exemplary embodiments of cosmetic compositions according to the present invention may also maintain a comfortable sensation and can lack a sticky sensation while exhibiting effective adhesion to the skin.

Further, exemplary embodiments of cosmetic compositions according to the present invention may maintain the aesthetic effect, particularly the gloss effect, over time.

Exemplary embodiments of cosmetic compositions according to the present invention may be able to impart a soft and smooth sensation and to maintain effective moisturizing.

In exemplary embodiments, cosmetic compositions according to the present invention exhibit good staying power with regard to external factors that may modify aesthetic properties, such as perspiration or consumption of a meal, in the case of a lipstick.

Staying power, gloss and comfort of a deposit of the composition according to exemplary embodiments can be assessed according to any method known to the skilled person. For example the staying power is assessed as described above.

Staying Power of the Composition

As used herein, the term “staying power” indicates the property of the cosmetic composition according to embodiments to transfer to a lesser extent onto objects with which the composition may come into contact, and the property of withstanding interaction with liquids, such as tears or perspiration, or contact with foods during a meal in the case of a lipstick, for example, and the property of not migrating beyond the initial outline of the makeup, for example, lipsticks, into the wrinkles and fine lines around the lips.

The cosmetic composition according to embodiments may exhibit the advantage of not transferring, at least in part, i.e. of leaving only the merest traces on certain substrates with which the cosmetic composition may be brought into contact, such as a glass, cup, cigarette, tissue, item of clothing, or the skin. The transfer of cosmetic compositions gives rise to poor staying power of the applied film, necessitating regular renewed application of the composition.

The cosmetic composition according to embodiments may exhibit good color fastness. The film of composition applied to the skin, lips and/or epidermal derivatives may generally be impaired during contact with liquids, such as water or beverages consumed, for example, during a meal, or alternatively oils, such as food oils or else sebum or else saliva. The color fastness may thus be characterized by the color fastness to water and/or the color fastness to oil.

The evaluation of the staying power properties of the cosmetic composition according to embodiments may be characterized by measurement of at least one of the following four parameters: transfer resistance, color fastness to water, color fastness to oil, and migration resistance.

The parameters of transfer resistance, color fastness to water, and color fastness to oil may be measured in succession according to the protocol described below.

The measurements are performed, for example, on the inner face of the forearm, which has been washed and allowed to dry naturally at ambient temperature for 5 minutes. The cosmetic composition to be tested, a lipstick for example, is applied to three areas of the inner surface of the forearm. The surface area of skin on which the measurements are performed must be at least greater than 1 cm². The measurements may be made on circular areas with a diameter of approximately 3 cm.

It is necessary for approximately the same amount of cosmetic composition to be applied to each of the three areas. This can be verified by measuring the weight of the cosmetic composition, the lipstick for example, after each of the applications, or by preparing equivalent amounts of sample to be tested beforehand. Generally speaking, for a surface area of 1 cm², an amount equal to approximately 2 mg is required (if the surface area has a diameter of 3 cm, then an amount of approximately 28 mg is required).

Following application of the cosmetic composition, the color, L₁*a₁*b₁*, is measured in each of the three areas, and the average value obtained corresponds to the initial color of the composition. The color measurement may be performed using a Minolta colorimeter of the CR200 or CR300 or CM500 or CM1000 or CM2000 series. The series CR200 Minolta calorimeter is used in some embodiments.

20 mg/cm² of water are added to each of the three areas to be tested (for surface areas with a diameter of approximately 3 cm, approximately 280 mg of water must be applied). Each of the areas to be tested is then massaged manually for a few seconds, such as from 2 to 5 seconds or from 2 seconds.

One thickness of a commercial white-paper handkerchief such as Kleenex, the L₀*a₀*b₀* color of which has been measured, is applied to each made-up area for approximately 5 seconds and at a force of approximately 100 g/f, which may be applied using a digital pressure dynamometer DPZ-5N from the manufacturer Imada Co. Ltd.

The transfer value T is obtained by subtracting the color of the white fabric measured before application to the area to be tested, L₀*a₀*b₀*, and the average color L₂*a₂*b₂* corresponding to the mean of the values obtained for each handkerchief after applying them to each area of the forearm covered with composition to be tested.

A determination is then made of the color difference ΔE(T) between the color of the handkerchief before and after application to the area of the forearm bearing the composition:
ΔE(T)={square root over ((L₂*−Lₒ*)²⁺(a₂*−aₒ*)²+(b₂*−bₒ*)})²

The lower the resulting ΔE(T) value, the more the cosmetic composition is considered to have a good level of transfer resistance.

In exemplary embodiments, the cosmetic compositions possess a transfer value ΔE(T) between about 0 and about 45, such as less than or equal to about 45, less than or equal to about 40, or less than or equal to about 35.

Subsequently, a measurement is made of the average color L₃*a₃*b₃* of the composition following application of the handkerchief.

The color fastness to water may be obtained after the transfer test has been carried out. It is equal, for example, to the color difference between the average initial color L₁*a₁*b₁* of the composition applied to the forearm and the average color L₃*a₃*b₃* of the area of the forearm bearing the composition following application of water and the handkerchief.
ΔE(W)={square root over ((L₃*−L₁*)²⁺(a₃*−a₁*)²+(b₃*−b₁*)})²

The lower the resulting value, the more the cosmetic composition is considered to possess good color fastness to water.

In exemplary embodiments, the color fastness value varies from about 0 to about 15. In exemplary embodiments, the cosmetic compositions possess a color fastness to water of less than or equal to about 15, such as less than or equal to 10, or less than or equal to 6.

The test for color fastness to oil is performed by applying, to the areas to be tested, approximately 20 mg/cm² of food-type oil on each area of the forearm (rapeseed oil, soya oil or sunflower oil), followed by manual massaging for a few seconds, such as from 2 to 5 seconds, or for 2 seconds. One thickness of a commercial white-paper handkerchief such as a Kleenex handkerchief is then applied to the area for approximately 5 seconds and at a force of approximately 100 g/f, which may be applied using a digital pressure dynamometer DPZ-SN from the manufacturer Imada Co. Ltd.

The color fastness to oil, 0, is equal to the difference between the average color L₄*a₄*b₄* of the composition remaining on the forearm after massaging with oil and application of the handkerchief, and the average color L₁*a₁*b₁* measured initially,
ΔE(O)={square root over ((L₃*−L₁*)²⁺(a₃*−a₁*)²+(b₃*−b₁*)})²

The test for color fastness to oil is a test that makes it possible to evaluate the hold of a cosmetic composition such as a lipstick during a meal.

In embodiments, the silicone polymer is such that, when present in sufficient amount in the composition, the color fastness to oil of a deposit of said composition, once the composition has been spread onto a substrate, is less than or equal to 25. In exemplary embodiments, the cosmetic compositions possess a color fastness to oil of less than or equal to 25, such as less than or equal to 10, or less than or equal to 8.

In particular embodiments, the silicone polymer is such that, when present in sufficient amount in the composition, the transfer value of said composition, once the composition has been spread onto a substrate, is less than or equal to about 35. The composition of embodiments may have a color fastness to water of less than 6 and a color fastness to oil of less than or equal to 8.

AVERAGE GLOSS OF THE COMPOSITION

In exemplary embodiments, the silicone polymer of general formula (I) is such that, when present in sufficient amount in the cosmetic composition, the average gloss at 60° of a deposit of the composition, once the composition has been spread onto a substrate, is greater than or equal to about 30 over about 100.

As used herein, the term “average gloss” indicates the gloss as may be measured using a gloss meter, conventionally, by the following method.

A gloss meter of Minolta GM268 type may be used. The measurements are carried out on test areas with a surface area of greater than 1 cm². In standard fashion, the surfaces onto which the cosmetic compositions under test are spread measure approximately 2.5 cm×4 cm.

The cosmetic composition for evaluation is applied to a synthetic surface, of Bioskin type. The amount of cosmetic composition to be applied is approximately 1 mg/cm².

The gloss value is obtained by measuring the reflectance at an angle of approximately 60°.

Five measurements per sample are necessary; the measurement having the highest value and the measurement having the lowest value are discarded, and an average is taken over the three remaining values measured.

The average gloss of the cosmetic compositions according to embodiments may be greater than or equal to about 30, such as greater than or equal to about 40, or greater than or equal to about 45.

Comfort

The comfort of the cosmetic composition according to embodiments may be evaluated by the test described below. In this test, the comfort of the composition is measured by a traction test on a latex strip. This test predicts the capacity, for a deposit of cosmetic composition, to withstand the flaking and peeling liable to occur following movements of the skin.

Samples of cosmetic composition are applied to surface areas of, for example, 2.54×2.54 cm, of a strip of latex 2.54 cm wide, obtained, for example, by cutting from the wrist area of a glove of Ansell Edmond Industrial Technicians type REF#390, size 9.

The amount of cosmetic composition to be deposited is such that the weight of the composition in terms of solids, should be approximately 20 mg.

The cosmetic composition is applied to the latex strip using a disposable lip brush, of the type, for example, produced by the company Femme Cosmetics, Inc., LA.

The samples thus prepared are left at ambient temperature for 24 hours.

The weight of the latex strip comprising the deposit of cosmetic composition is then measured (B). Subtracting the value of the weight of the latex strip devoid of cosmetic composition (A) from the value (B) thus measured should give the weight of dry film, and hence should therefore be approximately 20±2 mg.

The latex strip bearing the samples of cosmetic composition to be tested is subsequently pulled such that the area carrying the sample should reach a length of approximately 1.75 inches (4.445 cm).

The film fragments of the cosmetic composition that have detached from the latex strip are observed, and are then removed by brushing using the lip brush.

The weight of the latex strip comprising the remaining cosmetic composition is then measured (D).

The percentage weight loss of the film of the cosmetic composition is then calculated using the following equation:
Comfort index=[(D−A)/(B−A)]×100.

The measurements are repeated three times for each cosmetic composition tested. The comfort index of the composition according to the invention is equal to the average of these three measurements.

In exemplary embodiments, the silicone polymer of general formula (I) is such that, when present in sufficient amount in the cosmetic composition, the comfort index of a deposit of the composition on a substrate, is greater than or equal to about 90 over about 100, such as greater than about 95 over about 100.

Silicone Polymer of General Formula (I)

The silicone polymers in accordance with the silicone polymer of general formula (I) and possible for use in exemplary embodiments of cosmetic compositions are described in detail in patent application EP 1 213 316, which is incorporated by reference in its entirety herein.

The silicone polymers of general formula (I) may be used, in embodiments, as a surfactant and/or as an oily base.

Silicone polymers of general formula (I) that suitable for use in embodiments of the cosmetic compositions in accordance with the invention do not possess surface treatment functionality.

Introduced in sufficient quantity in embodiments, silicone polymers of general formula (I) may improve staying power, gloss and/or comfort of exemplary cosmetic compositions according to the invention.

In particular embodiments, the silicone polymers are represented by the general formula (I) below:
R¹ₐR²_{b}R³_{c}SiO_{(4-a-b-c)2} (I)
in which:
a) a ranges from 1 to 2.5; and b and c independently of one another range from 0.001 to 1.5;
b) R¹, which may be the same or different for each occurrence, is selected from:
   substituted and unsubstituted C₁ to C₃₀ alkyl radicals in which one or more fluorine atoms and/or amino and/or carboxyl groups may be substituents,
   aryl and aralkyl radicals,
   radicals of general formula (II):
      —C_{d}H_{2d}—O—(C₂H₄O)ₑ(C₃H₆O)_{f}R⁴ (II)
in which:
   R⁴ is a C₁ to C₃₀ hydrocarbon radical or a radical R⁵—(CO)—, and R⁵ is a C₁ to C₃₀ hydrocarbon radical, and
   d is an integer in the range of from 0 to 15 and e and f, independently of one another, are each integers in the range of from 0 to 50, and
   combinations thereof.
c) R² is represented by the general formula (III) below:
   -Q-O—X (I)
in which:
   Q is a divalent C₂ to C₂₀ hydrocarbon radical that can include at least one ether bond and/or at least one ester bond, and
   X is a polyhydroxylated hydrocarbon radical.
d) R³ is an organosiloxane group of general formula (IV): 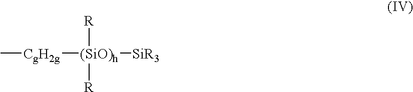
in which each R is independently selected from substituted and unsubstituted C₁ to C₃₀ alkyl radicals in which one or more fluorine atoms may be substituents, and aryl and aralkyl radicals,
g is an integer in the range of from 1 to 5 and h is an integer in the range of from 0 to 500.

When the radicals R are radicals selected from substituted and unsubstituted C₁ to C₃₀ alkyl radicals in which one or more fluorine atoms may be substituents, and from aryl radicals and aralkyl radicals, R has the same meaning as the radical R¹ as defined above.

It should be noted that the radicals R¹, R² and R³ of the silicone polymers of general formula (I), as defined above, are distributed randomly or statistically; that is, R¹, R² and R3 appear in the structure of the polymer without a determined order. Similarly, R¹, R² and R³ may respectively feature radicals of different kind in a compound of general formula (I).

In particular exemplary embodiments,

in a):
a is in the range of from 1.2 to 2.3, and b and c, independently of one another, are each in the range of from 0.05 to 1.

In b):
when R¹ is an alkyl radical, R¹ may be a C₁ to C₃₀ alkyl radical, for example, a C₁ to C₂₋₅ alkyl radical, such as a C₁ to C₂₋₀ alkyl radical, a C₁ to C₁₀ alkyl radical, a C₁ to C₆ alkyl radical, or a C₁ to C₄ alkyl radical. In some embodiments, R¹ may be a methyl, ethyl, n- or isopropyl, n- or iso- or tert-butyl, pentyl, hexyl, heptyl, octyl, nonyl, decyl, undecyl or lauryl radical. R¹ may also be a cycloalkyl radical, such as a cyclopropyl, a cyclobutyl, a cyclopentyl or a cyclohexyl. R¹ may also be a linear or branched, monounsaturated or polyunsaturated alkyl radical. R¹ may also be an alkyl radical substituted by one or more fluorine atoms, such as trifluoropropyl or heptadecafluorodecyl. In embodiments, R¹ may also be an alkyl radical substituted by one or more amino groups, such as 2-aminoethyl, 3-aminopropyl, and (2-ethylamino)-3-aminopropyl, or an alkyl group substituted by one or more carboxyl groups, such as 3-carboxypropyl.
R¹ may also be an aryl or aralkyl radical such as the phenyl radical, the tolyl radical, the benzyl radical and the phenethyl radical.
R¹ may also be an organic group represented by the general formula (II):
   —C_{d}H_{2d}—O—(C₂H₄O)ₑ(C₃H₆O)_{f}R⁴ (II)

According to some exemplary embodiments, R¹ may be a hydroxylated radical or a radical obtained from the addition reaction of a saturated or unsaturated, linear or branched alkenyl ether in which d=0 and which is therefore of formula:
—O—(C₂H₄O)ₑ(C₃H₆O)_{f}R⁴

In embodiments in which e and f are zero, R¹ is an alkoxy group having from 4 to 30 carbon atoms, for example a C₄ to C₁₀ lower alkoxy radical, such as butoxy or pentoxy, or a C₁₁ to C₃₀ higher alkoxy radical, such as oleoxy, stearoxy, viz., for example, cetyl alcohol, oleyl alcohol and stearyl alcohol, or a radical obtained from an acid or a fatty acid, such as acetic acid, lactic acid, butyric acid, oleic acid, stearic acid and behenic acid.

When e and f are greater than 1, R¹ is a hydroxyl radical originating from the addition reaction of an alkylene oxide.

In embodiments in which e and fare zero d maybe 3, 5 or 11, in which R¹, depending on the nature of the substituent R⁴, is an allyl ether, pentenyl ether or undecenyl ether radical or an allyl stearyl ether, pentenyl behenyl ether or undecenyl oleyl ether radical.

When e and/or f are not zero, an alkoxy radical and an ester radical are present via a polyoxyalkylene group in embodiments.

Regardless of the values of e and f d may be, in embodiments, within the range of from 3 to 5.

According to an exemplary embodiment, the radical R¹ may be any one of the above-defined radicals or a combination of two or more of these radicals.

In exemplary embodiments, R¹ is an alkyl radical selected from the methyl radical, the lauryl radical, and combinations thereof.

In some exemplary embodiments, R¹ represents two or more radicals in a single general formula (I), a methyl radical and a lauryl radical, for example, these radicals appear in the structure at random and with a frequency that is specific to them.

In particular embodiments, methyl radicals are at least 50% of the radicals R¹, at least 70% of the radicals R¹, or 100% of the radicals R¹ are methyl radicals.

In c):
Q maybe a divalent hydrocarbon radical selected from:
—(CH₂)₂—, —(CH₂)₃—, —CH₂CH(CH₃)—CH₂, —(CH₂)₄—, —(CH₂)₅—, —(CH₂)₆—, —(CH₂)₇—, —(CH₂)₈—, —(CH₂)₉—, —(CH₂)₁₀, —(CH₂)₁₁—, —(CH₂)₂—CH(CH₂CH₂CH₃)—, —CH₂—CH(CH₂CH₃)—, —(CH₂)₃—O—(CH₂)₂—, —(CH₂)₃—O—(CH₂)₂—O—(CH₂)₂—, —(CH₂)₃—O—CH₂CH(CH₃)— and —CH₂—CH(CH₃)—COO(CH₂)₂—.

In exemplary embodiments, Q may be a divalent radical selected from —(CH₂)₂— and —(CH₂)₃—.
X may be, in exemplary embodiments, a polyhydroxylated hydrocarbon radical containing at least two hydroxyl residues, such as a hydrocarbon group selected from glyceryl derivatives and saccharide derivatives.

The glycerol residues may be compounds having the following formulae, in which Q has the same meaning as in the general formula (III), and s and t are integers in the range of from 1 to 20, for example from 1 to 15, from 1 to 10, or from 1 to 5.

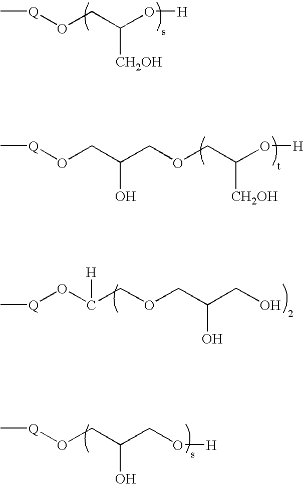

In the above formulae, one or more hydroxyl groups may be replaced by alkoxy groups or ester groups.

The saccharide radicals, which can be used in general formula (III) of embodiments, may be of monosaccharide type, such as glycosyl, mannosyl, galactosyl, ribosyl, arabinosyl, xylosyl or fructosyl groups, of oligosaccharide type, such as maltosyl, cellobiosyl, lactosyl or maltotriosyl, or a polysaccharide type, such as cellulose or starch.

In particular embodiments, the saccharide groups may be of monosaccharide or oligosaccharide type.

In d):
each of the radicals R may represent, independently of one another, a radical chosen from C₁ to C₂₀, such as C₁ to C₁₀, or C₁ to C₆ alkyl radicals, which may be substituted with one or more fluorine atoms. When the radicals R represent a radical chosen from alkyl radicals as defined above, optionally substituted with one or more fluorine atoms, the radicals R have the same meaning as the radical R¹ as defined above.
g, according to an exemplary embodiment, is 2.
h, according to an exemplary embodiment, is within the range of from 1 to 50.

However, in certain exemplary embodiments, the silicone polymer of general formula (I) is such that, when the radical R² is represented by the general formula (IIIA):
—C₃H₆O[CH₂CH(OH)CH₂O]ₙH (IIIA)
in which n ranges from 1 to 5, the radical R¹ is not a C₁₂ alkyl radical.

According to an exemplary embodiment, the silicone polymer of general formula (I) is further defined as follows:
a ranges from 1 to 1.4 and b and c, independently of one another, range from 0.02 to 0.03, and
R¹ is a C₁ to C₁₀, such as C₁ to C₆, or C₁ to C₄ alkyl radical.
   R² is represented by the formula (IIIA):
      —C₃H₆O[CH₂CH(OH)CH₂O]ₙH (IIIA)
in which:
   n is in a range from 1 to 5, and
   R³ is represented by the formula (IVA):
      —C₂H₄(CH₃)₂SiO[(CH₃)₂SiO]ₘSi(CH₃)₃ (IVA)
in which:
   m is in a range from 3 to 9.

According to additional exemplary embodiments, the silicone polymer of general formula (I), which can be used in the cosmetic compositions according to embodiments of the present invention, is such that:
a ranges from 1 to 1.4 and b and c, independently of one another, range from 0.02 to 0.04,
R¹ is a methyl radical,
R² is represented by the formula (“IA) in which n ranges from 1 to 5, and
R³ is represented by the formula (IVA) in which m ranges from 3 to 9.

In exemplary embodiments, the silicone polymer of general formula (I) may be chosen from polyglyceryl-3 polymethylsiloxyethyl dimethicone, laurylpolyglyceryl-3 polymethylsiloxyethyl dimethicone and polyglyceryl-3 disiloxane dimethicone, whose formulae are, respectively:
polyglyceryl-3 polymethylsiloxyethyl dimethicone (formula (V)): 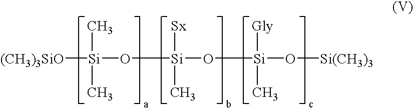
in which:
Sx: —C₂H₄[(CH₃)₂SiO]ₘSi(CH₃)₃
Gly: —C₃H₆O[CH₂—CH(OH)CH₂O]ₙH
with a=1-1.4, b=0.02-0.04, c=0.02-0.04, m=3-9, n=1-5,
   lauryl polyglyceryl-3 polymethylsiloxyethyl dimethicone (formula (VI)): 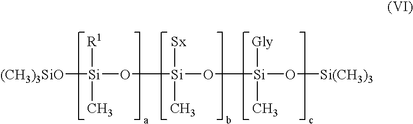
in which Sx, Gly, a, b, c, m and n have the same meaning as above and R¹ is either a methyl radical or a lauryl radical.
   Polyglyceryl-3 disiloxane dimethicone (formula (VII)): 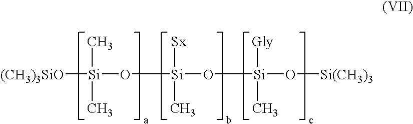
in which Gly, a, b, c, m and n have the same meaning as above, and
Sx: —O(CH₃)₂SiO—Si(CH₃)₃

The silicone polymer of general formula (I) may be present in the cosmetic compositions in accordance with exemplary embodiments of the present invention in a proportion of from about 0.1% to about 40% by weight, such as from 0. about 5% to about 30% by weight, from about 1% to about 25% by weight, from about 5% to about 20% by weight, or from about 7% to about 15% by weight, relative to the total weight of the composition.

The silicone polymer of general formula (I) may be employed in a free form in exemplary embodiments.

As used herein, the term “free form” encompasses forms of silicone polymers of general formula (I) in which the polymer is not employed in a form in which the polymer is combined with or adsorbed on another material, such as, for example, in EP 1 416 016 and EP 1 424 373, where the polymer is present in the form of a coating of a powder or a colorant for the purpose of blocking the surface activity of the particles making up the corresponding powder.

According to an exemplary embodiment, the silicone polymer of general formula (I) may be chosen from polymers sold by SHIN-ETSU under the references KF6100®, KF6104® and KF6105®.

The polymer sold under the reference KF6104® may be used in exemplary embodiments of the cosmetic compositions.

The KF6104® polymer, sold by SHIN-ETSU, may be particularly suitable for preparing exemplary embodiments of cosmetic compositions in accordance with the invention that exhibit improved staying power on keratin materials without detriment to the gloss or that may exhibit improved average gloss.

Short Hydrocarbon Esters

As used herein, the term “short hydrocarbon ester” encompasses hydrocarbon esters containing less than 40 carbon atoms.

The cosmetic compositions of embodiments may comprise at least one short hydrocarbon ester.

The esters in accordance with embodiments may be monoesters, diesters or polyesters. In particular embodiments, the esters are monoesters, or esters bearing only one ester function. The esters of embodiments may be linear, branched or cyclic and may be saturated or unsaturated. In particular embodiments, the esters may be branched and saturated. Esters of embodiments may also be volatile or non-volatile.

In particular embodiments, the hydrocarbon esters may correspond to the formula RCOOR′ in which RCOO represents a fatty acid residue containing 2 to 28 carbon atoms and R′ represents a hydrocarbon chain containing 1 to 28 carbon atoms. In certain embodiments, the groups R and R′ are such that the corresponding ester is non-volatile.

In exemplary embodiments, the mono-, di- or polyester hydrocarbon esters that can be used in the cosmetic compositions contain less than 40 carbon atoms and more than 10 carbon atoms.

Such non-volatile esters may be C₁₀ to C₂₅ esters, such as C₁₄ to C₂₂ esters. The non-volatile hydrocarbon esters of embodiments may be chosen from esters of C₂ to C₁₈ acids, of C₂ to C₂₀ alcohols, of C₂ to C₈ polyols, and of mixtures thereof.

The non-volatile hydrocarbon esters of embodiments may contain advantageously less than 22 carbon atoms.

According to particular embodiments, when the hydrocarbon ester contains less than 22 carbon atoms, the hydrocarbon ester is not a volatile oil.

In embodiments, the hydrocarbon esters may be chosen from, for example, esters of neopentanoic acid, for instance isodecyl neopentanoate, isotridecyl neopentanoate, isostearyl neopentanoate and octyldodecyl neopentanoate, esters of isononanoic acid, for instance isononyl isononanoate, octyl isononanoate, isodecyl isononanoate, isotridecyl isononanoate and isostearyl isononanoate, esters of isopropyl alcohol, such as isopropyl myristate, isopropyl palmitate, isopropyl stearate or isostearate, cetyl octanoate, tridecyl octanoate, 2-ethylhexyl 4-diheptanoate and palmitate, alkyl benzoate, polyethylene glycol diheptanoate, propylene glycol di-2-ethylhexanoate, and mixtures thereof. The hydrocarbon esters of embodiments may also be chosen from synthetic esters, for example, of fatty acids, such as purcellin oil, isopropyl myristate, ethyl palmitate and octyl stearate; hydroxylated esters such as isostearyl lactate, octyl hydroxystearate, diisopropyl adipate, fatty alcohol heptanoates, octanoates and decanoates, and mixtures thereof.

Isononyl isononanoate, isotridecyl isononanoate, stearyl heptanoate and mixtures thereof may be used in exemplary embodiments. According to particular embodiments, the short hydrocarbon esters may be a mixture of isononyl isononanoate, isotridecyl isononanoate and stearyl heptanoate.

This or these hydrocarbon ester(s) may be used in exemplary compositions in a proportion of from about 5% to about 90%, such as from about 10% to about 60% or from about 20% to about 50%, by weight relative to the total weight of the composition.

Physiologically Acceptable Medium

As used herein, a “physiologically acceptable medium” encompasses any non-toxic medium that can be applied to the skin, lips or keratin materials of human beings. The physiologically acceptable medium generally can be adapted to the nature of the substrate to which the composition is to be applied, and also to the form in which the composition is to be packaged.

The physiologically acceptable medium of embodiments may comprise an aqueous phase and/or a fatty phase.

In exemplary embodiments, the aqueous phase or the fatty phase may form the continuous phase of the composition.

Exemplary embodiments of cosmetic compositions in accordance with the present invention may be presented in the form of an emulsion, in which the silicone polymer of general formula (I), as defined above, may have the function of a surfactant.

For the purposes of the present invention the emulsions contain a lipophilic phase and a hydrophilic phase, the latter not systematically being water.

Thus, the cosmetic compositions of exemplary embodiments in accordance with the invention may be in the form of a water-in-oil, oil-in-water, multiple or anhydrous emulsion.

In particular embodiments, the cosmetic compositions in accordance with the invention may be in the form of an anhydrous emulsion.

In exemplary embodiments, the cosmetic compositions may possess, for example, a continuous fatty phase that may contain less than about 10% by weight of water, for example, less than about 5% by weight of water.

The cosmetic compositions according to exemplary embodiments may be anhydrous: that is, exemplary cosmetic compositions may contain less than about 5%, such as less than about 3%, less than about 2%, or less than about 1% of water relative to the total weight of the composition. Such exemplary cosmetic compositions may then be in the form in particular of oily gels, oily liquids, pastes or sticks or may be in the form of a vesicular dispersion containing ionic and/or non-ionic liquids.

Fatty phase

The cosmetic compositions of embodiments may include a fatty phase that includes oils and fats which are solid at ambient temperature (20-25° C.) and atmospheric pressure.

As used herein, an oil encompasses any fatty substances that are in liquid form at ambient temperature (20-25° C.) and at atmospheric pressure. The liquid fatty phase may also, besides the oils, contain other compounds dissolved in the oils, such as gelling and/or structuring agents.

The cosmetic compositions of embodiments may include at least one, such as at least two, oil(s) other than the ester described above.

The oil or oils may be present in a proportion of from about 0.1% to about 99% by weight, such as about 1% to about 90% by weight, from about 5% to about 70% by weight, from about 10% to about 60% by weight, or from about 20% to about 50% by weight, relative to the total weight of the cosmetic composition according to embodiments.

The oils suitable for preparing cosmetic compositions according to embodiments may be volatile or non-volatile, silicone or non-silicone oils.

As used herein, “volatile oil” encompasses oils (or non-aqueous medium) that are capable of evaporating on contact with the skin in less than one hour at ambient temperature and atmospheric pressure. The volatile oil of embodiments may be a volatile cosmetic oil that are liquid at ambient temperature, and that may have a non-zero vapour pressure, at ambient temperature and atmospheric pressure. In particular embodiments, the volatile oil may have a vapour pressure ranging from about 0.13 Pa to about 40,000 Pa (about 10⁻³ to about 300 mmHg), such as from about 1.3 Pa to about 13,000 Pa (about 0.01 to about 100 mmHg), or from about 1.3 Pa to about 1300 Pa (about 0.01 to about 10 mmHg).

As used herein, “non-volatile oil” encompasses oils having a vapor pressure of less than about 0.13 Pa. The volatile or non-volatile oils of embodiments may be hydrocarbon oils, such as those of animal or plant origin, synthetic oils, silicone oils, fluoro oils, or mixtures thereof.

As used herein, “silicone oil” encompasses oils containing at least one silicon atom, such as those containing at least one Si—O group.

As used herein, “hydrocarbon oil” encompasses oils containing principally hydrogen and carbon atoms and optionally oxygen, nitrogen, sulphur and/or phosphorous atoms.

The volatile hydrocarbon oils of embodiments may be chosen from hydrocarbon oils having 8 to 16 carbon atoms, such as branched C₈-C₁₆ alkanes (also known as isoparaffins), for instance isododecane (also known as 2,2,4,4,6-pentamethylheptane), isodecane, isohexadecane, and, for example, the oils sold under the trade names ISOPARS® or PERMETHYLS®.

As volatile oils of embodiments, volatile silicones may be used. Such volatile silicones include, for example, volatile linear or cyclic silicones, such as those having a viscosity ≦8 centistokes (8×10⁻⁶ m²/s), and those having 2 to 10 silicon atoms, for example, silicones having 2 to 7 silicon atoms, and silicones optionally containing alkyl or alkoxy groups having 1 to 10 carbon atoms. In embodiments, volatile silicone oils may be chosen from, for example, dimethicones with a viscosity of 5 and 6 cSt, octamethyl-cyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

Volatile fluoro oils, such as nonafluoromethoxybutane or perfluoromethyl-cyclopentane, and mixtures thereof, may also be used in embodiments.

The fatty phase of the cosmetic compositions according to embodiments may also comprise at least one volatile oil.

According to particular embodiments, volatile oil may be present in amounts that are less than 30% by weight, such as less than about 15%, less than about 10%, or less than about 5%, by weight, relative to the total weight of the composition.

In other embodiments, the cosmetic compositions are free from volatile oils.

The fatty phase of the cosmetic compositions according to exemplary embodiments may also comprise at least one non-volatile oil.

The non-volatile oils of embodiments may be chosen from hydrocarbon oils, which may be optionally fluorinated, and/or non-volatile silicone oils.

The non-volatile hydrocarbon oil of embodiments may be chosen from, for example:
hydrocarbon oils of animal origin;
hydrocarbon oils of plant origin, such as phytostearyl esters, for instance phytostearyl oleate, phytostearyl isostearate and lauroyl/octyldodecyl/phytostearyl glutamate (Ajinomoto, Eldew PS203), triglycerides consisting of fatty acid glycerol esters, in which the fatty acids may have varied linear or branched and saturated or unsaturated chain lengths from C₄ to C₂₄; and in embodiments, the hydrocarbon oils of plant origin may be chosen from heptanoic or octanoic triglycerides, wheatgerm oil, sunflower oil, grapeseed oil, sesame oil, corn oil, apricot oil, castor oil, shea oil, avocado oil, olive oil, soya oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; shea butter; or else caprylic/capric acid triglycerides such as those sold by Stearineries Dubois or those sold under the names Miglyol 810®, 812® and 818® by Dynamit Nobel;
synthetic esters having 10 to 40 carbon atoms;
linear or branched hydrocarbons of mineral or synthetic origin, such as petroleum jelly, polydecenes, hydrogenated polyisobutene such as parleam, and squalane, and mixtures thereof;
polyol esters and pentaerythritol esters, such as dipentaerythritol tetrahydroxystearate/tetraisostearate;
esters of diol dimers and of diacid dimers, such as the products LUSPLAN DD-DA5® and LUSPLAN DD-DA7®, sold by Nippon Fine Chemical and described in FR 0302809, the content of which is incorporated herein by reference in its entirety;
fatty alcohols that are liquid at ambient temperature, containing a branched and/or unsaturated carbon chain having 12 to 26 carbon atoms, such as 2-octyldodecanol, isostearyl alcohol, oleyl alcohol, 2-hexyldecanol, 2-butyloctanol and 2-undecylpenta-decanol;
higher fatty acids, such as oleic acid, linoleic acid, linolenic acid and mixtures thereof; and
dialkyl carbonates, the two alkyl chains possibly being identical or different, such as the dicaprylyl carbonate sold under the name CETIOL CC®, by Cognis.

The non-volatile silicone oils that can be used in compositions according to embodiments may be chosen from non-volatile polydimethylsiloxanes (PDMS), polydimethylsiloxanes containing pendent alkyl or alkoxy groups and/or alkyl or alkoxy groups that are at the ends of a silicone chain, these groups each having 2 to 24 carbon atoms, phenyl silicones, for instance phenyltrimethicones, phenyldimethicones, phenyl trimethylsiloxy diphenylsiloxanes, diphenyl dimethicones, diphenyl methyldiphenyl trisiloxanes, and 2-phenylethyl trimethylsiloxysilicates, and mixtures thereof.

In exemplary embodiments, the non-volatile oil are chosen from hydrogenated polyisobutene, isostearyl heptanoate, isononyl isononanoate, isotridecyl isononanoate, diisostearyl malate, dipentaerythritol tetrahydroxystearate/tetraisostearate, 2-octyldodecanol, and mixtures thereof.

According to particular embodiments, the non-volatile oil may be a mixture of hydrogenated polyisobutene, isostearyl heptanoate, isononyl isononanoate, isotridecyl isononanoate, diisostearyl malate, dipentaerythritol tetrahydroxystearate/tetraisostearate and 2-octyldodecanol.

The non-volatile oils of embodiments may be present amounts ranging from about 20% to about 99% by weight, such as from about 30% to about 80% by weight or from about 40% to about 80% by weight, relative to the total weight of the composition.

According to particular embodiments, the liquid fatty phase of the cosmetic compositions is a silicone oil present in an amount ranging from about 0 to about 90% by weight, such as from about 0.1% to about 80% by weight or from about 2% to about 80% by weight, relative to the total weight of the composition.

According to embodiments, the silicone oil may be present in the cosmetic compositions in a weight ratio, relative to the silicone polymer of general formula (I), of from about 80:1, such as from about 60:1 or from about 40:1.

The liquid fatty phase of some embodiments may be thickened, gelled or structured by incorporation therein of a gelling agent for the liquid fatty phase, as defined below.

The compositions according to embodiments may also include at least one compound chosen from waxes, pasty fatty substances and mixtures thereof.

The wax of exemplary embodiments may be solid at ambient temperature (25° C.), may have a reversible solid/liquid state change, may have a melting temperature of greater than 30° C. such as up to 200° C., may have a hardness of more than 0.5 MPa, and may exhibit in the solid state an anisotropic crystalline organization. In embodiments, the wax may be hydrocarbon-, fluorine- and/or silicone-based and may be animal, vegetable, mineral or synthetic in origin. The wax of embodiments may be chosen, for example, from beeswax, carnauba wax, candelilla wax, paraffin waxes, hydrogenated castor oil, synthetic waxes such as polyethylene waxes (including those having a molecular weight of between 400 and 600) or Fischer-Tropsch waxes, silicone waxes such as alkyl- or alkoxy-dimethicones having 16 to 45 carbon atoms, ceresines or ozokerites, such as, for example, isoparaffins whose melting point is less than 40° C., such as EMW-0003, sold by NIPPON SEIROU, α-olefin oligomers, such as the PERFORMA V® polymers 825, 103 and 260, sold by NEW PHASE TECHNOLOGIES; ethylene-propylene copolymers, such as PERFORMALENE® EP 700, and microcrystalline waxes whose melting point is greater than 85° C., such as the HI-MIC” products 1070, 1080, 1090 and 3080, sold by NIPPON SEIROU, and mixtures thereof.

In exemplary embodiments, the wax is chosen from polyethylene waxes, candelilla wax and mixtures thereof.

According to particular embodiments, the cosmetic compositions include a mixture of polyethylene wax and candelilla wax.

According to particular embodiments, the wax used in the cosmetic compositions in accordance with embodiments is present in an amount varying from about 1.5% to about 20%, such as from about 3% to about 15%, from about 5% to about 10%, or from about 6.5% to about 8.5% by weight relative to the total weight of the composition.

The cosmetic compositions in accordance with embodiments may also include at least one pasty compound.

As used herein, the term “pasty” indicates fatty compounds that exhibit reversible solid/liquid state change and at a temperature of 23° C. have liquid and solid fractions. For example, polyvinyl laurate is pasty.

The pasty compound of embodiments may exhibit a hardness at 20° C. ranging from about 0.001 to about 0.5 MPa, such as from about 0.002 to about 0.4 MPa.

Pasty compounds that can be used in embodiments include, for example, lanolins and lanolin derivatives such as acetylated lanolins, oxypropylenated lanolins or isopropyl lanolate, and mixtures thereof; esters of fatty alcohols or acids, for example those having 20 to 65 carbon atoms, such as triisostearyl citrate or cetyl citrate; arachidyl propionate; polyvinyl laurate; cholesterol esters such as triglycerides of vegetable origin, for instance hydrogenated vegetable oils or hydrogenated castor oil derivatives, such as THIXINR® from Rheox; viscous polyesters; and mixtures thereof.

In some embodiments, polyesters resulting from the esterification of a carboxylic acid and an aliphatic hydroxycarboxylic acid ester may be included, such as, for example, RISOCAST® da-1 (ester obtained from the esterification reaction of hydrogenated castor oil with dilinoleic acid in proportions of 2 to 1) and RISOCAST® da-h (ester resulting from the esterification of hydrogenated castor oil with isostearic acid in proportions of 4 to 3), which are sold by the Japanese company KOKYU ALCOHOL KOGYO.

Hydrogenated cocoglycerides may also be used as pasty compounds of embodiments.

In addition, the pasty compounds of embodiments may be chosen from pasty silicone compounds such as the high molecular weight polydimethylsiloxanes (PDMS), such as those having pendent chains of the alkyl or alkoxy type having 8 to 24 carbon atoms, and a melting point of 20-55° C., such as stearyldimethicones, including those sold by DOW CORNING under the trade names DC2503® and DC255 14®; and mixtures thereof.

Aqueous Phase

In exemplary embodiments, cosmetic compositions may include at least one aqueous medium, constituting an aqueous phase, which may form the continuous phase of the composition.

The aqueous phase may be composed essentially of water.

The aqueous phase may be composed, in embodiments, of a mixture of water and a water-miscible organic solvent (with a miscibility in water of more than 50% by weight at 25° C.). The water-miscible organic solvent of embodiments, may be chosen from lower monoalcohols having 1 to 5 carbon atoms, for instance ethanol, isopropanol; glycols having 2 to 8 carbon atoms, for instance propylene glycol, ethylene glycol, 1,3-butylene glycol and dipropylene glycol; C₃-C₄ ketones; and C₂-C₄ aldehydes.

The aqueous phase (water and, optionally, the water-miscible organic solvent) may be present in embodiments in an amount ranging from 0.1% to 40% by weight, such as from 0.1% to 20% by weight or from 0.1% to 10% by weight, relative to the total weight of the composition.

Polyols

According to embodiments, the cosmetic compositions may also include at least one polyol or polyhydric alcohol.

As used herein, the terms “polyhydric alcohol” or “polyol”, encompass any organic molecule containing at least two free hydroxyl groups.

Polyhydric alcohols that may be used in cosmetic compositions according to embodiments include those having, in particular, 2 to 20 carbon atoms, such as 2 to 10 carbon atoms or 2 to 6 carbon atoms.

In exemplary embodiments, the polyol may be chosen, for example, from glycerol; propylene glycol; butylene glycol; pentylene glycol; hexylene glycol; dipropylene glycol; diethylene glycol; sorbitol; hydroxypropylsorbitol, 1,2,6-hexanetriol; glycol ethers, including those glycol ethers having 3 to 16 carbon atoms such as the (C₁-C₄)alkyl ethers of mono-, di- or tripropylene glycol and the (C₁-C₄)alkyl ethers of mono-, di- or triethylene glycol; and mixtures thereof.

Silicone Compounds

As used herein, the term “silicone compound” encompasses compounds, other than the silicon polymer of general formula (1), that include at least one silicon atom.

Compositions according to exemplary embodiments may include, in addition to the polymer of general formula (I), a silicone compound chosen from the aforementioned silicone oils, silicone gums, aforementioned silicone resins, silicone elastomers, and mixtures thereof.

In embodiments in which the silicone compound is a silicone oil, the silicone oils may be as defined above.

In embodiments in which the silicone compound is a silicone resin, the silicone resins in question may be as described in application FR 0 450 540, the content of which is incorporated herein by reference.

The cosmetic compositions of embodiments may also include a silicone gum.

Silicone gums, in embodiments, are fluid or solid compounds having a weight-average molecular mass of greater than or equal to 200,000 g/mol, such as from about 200,000 to about 4,000,000 or from about 200,000 to about 2,000,000 g/mol.

The viscosity of fluid silicone gums used in embodiments may vary in the range from about 1000 to about 10,000,000 cSt, such as from about 100,000 to about 1,000,000 cSt, or from about 300,000 to about 700,000 cSt, as measured in accordance with the standard ASTM D-445.

The silicone gum of embodiments may be an ungrafted polymer, i.e. a polymer obtained by polymerizing at least one monomer without subsequent reaction of the side chains with another chemical compound. The silicone gum may be chosen, in embodiments, from dimethiconols, fluorosilicones, dimethicones and mixtures thereof. The silicone gum of particular embodiments may be a homopolymer.

In some embodiments, the silicone gum may be representable by formula (VIII): 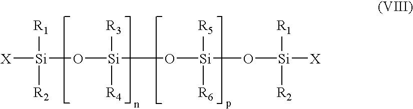
in which:
R₁, R₂, R₅ and R6 are, independently of one another, chosen from alkyl radicals having 1 to 6 carbon atoms that are each optionally substituted by at least one fluorine atom,
R₃ and R4 are, together or separately, chosen from alkyl radicals having 1 to 6 carbon atoms and aryl radicals,
X is chosen from alkyl radicals having 1 to 6 carbon atoms, hydroxyl radicals, vinyl or allyl radicals, and alkoxy radicals having 1 to 6 carbon atoms,
n and p are chosen to provide a silicone compound having a weight-average molecular mass of greater than or equal to 200,000 g/mol.
p may be 0 in some embodiments.

Such exemplary polymers of formula (VIII) include dimethiconols, which are polymers in which R¹ to R6 are methyl groups and X is a hydroxyl group. Examples include polymers of formula (IX) in which p=0 and n is between about 2000 and about 40 000, such as between about 3000 and about 30 000. Exemplary polymers may also have a molecular mass of between about 1,500,000 and about 2,000,000 g/mol.

According to embodiments, the silicone gum may be the dimethiconol sold by DOW CORNING in a polydimethylsiloxane (5 cSt) under reference D2-9085, the viscosity of the mixture being 1550 cSt, the dimethiconol sold by DOW CORNING in a polydimethylsiloxane (5 cSt) under reference DC 1503, or the dimethiconol (with a molecular weight of 1,770,000 g/mol) sold by DOW CORNING under reference Q2-1403 or Q2-1401, the viscosity of the mixture being 4000 cSt.

Silicone gums that can be used in accordance with embodiments include silicone gums in which:
substituents R¹ to R⁶ and X are methyl groups, such as that sold under the name SE30 by GENERAL ELECTRIC, and that sold under the name AK 500 000 by WACKER,
substituents R¹ to R6 and X are methyl groups and p and n are such that the molecular weight is 250,000 g/mol, such as that sold under the name SILBIONE 70047 V by RHODIA,
substituents R¹ to R6 are methyl groups, and X is a hydroxyl group, such as that sold under the name Q2-1401 or Q2-1403 by DOW CORNING,
substituents R₁, R₂, R₅, R₆ and X are methyl groups, substituents R₃ and R₄ are aryl groups, and n and p are such that the molecular weight of the polymer is 600,000 g/mol, such as that sold under the name 761 by RHODIA-CHIMIE.

Silicone gum can be introduced into the composition according to embodiments in the form of a mixture with a silicone oil, the viscosity of said oil varying from about 0.5 to about 10,000 cSt, such as from about 0.5 to about 500 cSt, or from about 1 to about 10 cSt.

In embodiments, the silicone oil in a mixture with the silicone gum may be chosen from polyalkylsiloxanes, polyarylsiloxanes, polyalkylarylsiloxanes and mixtures thereof. The liquid silicone may be a volatile silicone such as a cyclic polydimethylsiloxane containing 3 to 7 —(CH₃)₂SiO— units.

The silicone oil of embodiments may also be a non-volatile polydimethylsiloxane silicone, such as those having a viscosity of between 8 and 10 000 cSt, for instance 10 cSt, for example the silicone sold under reference DC 200 by DOW CORNING.

The proportion of the silicone gum in the gum/oil mixture may be between about 10/90 and about 20/80 in embodiments. The viscosity of the gum/oil mixture may be between about 1000 and about 10,000 cSt in embodiments.

High molecular weight dimethicones according to embodiments include the dimethicones described in U.S. Pat. No. 4,152,416 and are sold, for example, under references SE30, SE33, SE 54 and SE 76.

Dimethicones according to embodiments may also be compounds of the above formula in which R¹ to R₆ and X are methyls and p=0. The molecular weight of such compounds may be between about 200,000 and about 300,000, such as between about 240,000 and about 260,000 g/mol.

Dimethicones according to embodiments also may include polydimethylsiloxanes, (polydimethylsiloxane)-(methylvinylsiloxane) copolymers, poly(dimethyl-siloxane)(diphenyl)(methylvinylsiloxane) copolymers, and mixtures thereof.

The fluorosilicone gums of high molecular weight according to embodiments may have molecular weights varying from about 200,000 to about 300,000 g/mol, such as from about 240,000 to about 260,000 g/mol.

The silicone compound that can be used in the cosmetic compositions of embodiments may also be a silicone elastomer. The silicone elastomer may be, in embodiments, a polyglycerolated silicone elastomer, which is different from the silicone polymer of general formula (I).

The polyglycerolated silicone elastomer that can be used in formulating the compositions according to exemplary embodiments may be an elastomeric crosslinked organopolysiloxane obtainable by crosslinking addition reaction of a diorganopolysiloxane containing at least one hydrogen bonded to the silicon and of polyglycerolated compounds having ethylenically unsaturated groups, optionally in the presence of a platinum catalyst.

In particular embodiments, the elastomeric crosslinked organopolysiloxane is obtained by crosslinking addition reaction (A) of a diorganopolysiloxane containing at least two hydrogens each bonded to a silicon, and (B) of glycerolated compounds having at least two ethylenically unsaturated groups, optionally in the presence (C) of a platinum catalyst.

In further particular embodiments, the organopolysiloxane may be obtained by reacting a polyglycerolated compound containing dimethylvinylsiloxy end groups and a methylhydropolysiloxane containing trimethylsiloxy end groups, in the presence of a platinum catalyst.

Compound (A) is the base reactant for the formation of elastomeric organopolysiloxane and the crosslinking is affected by addition reaction of the compound (A) with the compound (B) in the presence of the catalyst (C).

Compound (A) may be an organopolysiloxane having at least two hydrogen atoms bonded to different silicon atoms in each molecule.

Compound (A) may have any molecular structure, for example, a linear chain structure, a branched chain structure or a cyclic structure.

Compound (A) may have a viscosity at 25° C. ranging from 1 to 50,000 centistokes, in order to be well miscible with the compound (B), in embodiments.

The organic groups bonded to silicon atoms of the compound (A) in embodiments may be alkyl groups having 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl and 3,3,3-trifluoro-propyl; aryl groups such as phenyl, tolyl and xylyl; substituted aryl groups such as phenylethyl; and substituted monovalent hydrocarbon groups such as an epoxy group, a carboxylate ester group or a mercapto group. In particular embodiments, the organic group is chosen from methyl, phenyl and lauryl groups.

Compound (A) may thus be chosen in embodiments from methylhydropolysiloxanes containing trimethylsiloxy end groups, dimethylsiloxane-methylhydrosiloxane copolymers containing trimethylsiloxy end groups, cyclic dimethyl-siloxane-methylhydrosiloxane copolymers and dimethylsiloxane-methylhydrosiloxane-laurylmethyl-siloxane copolymers containing trimethylsiloxy end groups.

Compound (B) may be a polyglycerolated compound corresponding to the formula (B′) below:
CₘH₂ₘ₋₁—O-[Gly]n—CₘH₂ₘ₋₁(B′)
in which m is an integer ranging from 2 to 6; n is an integer ranging from 2 to 200, for example from 2 to 100, from 2 to 50, from 2 to 20, ranging from 2 to 10, or ranging from 2 to 5; and in particular embodiments, n=3; Gly denotes:
   —CH₂—CH(OH)—CH₂—O— or —CH₂—CH(CH₂OH)—O—

In exemplary embodiments, the sum of the number of ethylenic groups per molecule of compound (B) and of the number of hydrogen atoms bonded to silicon atoms per molecule of the compound (A) is at least 4.

Compound (A), in embodiments, may be added in an amount such that the molecular ratio between the total amount of hydrogen atoms bonded to silicon atoms in the compound (A) and the total amount of all of the ethylenically unsaturated groups in the compound (B) is within the range from 1/1 to 20/1.

Compound (C) is the catalyst of the crosslinking reaction of embodiments, and may be chosen from chloroplatinic acid, chloroplatinic acid-olefin complexes, chloroplatinic acid-alkenylsiloxane complexes, chloroplatinic acid-diketone complexes, platinum black, and platinum on a support.

The catalyst (C) by be added, in embodiments, in amounts of from about 0.1 to about 1000 parts by weight, such from about 1 to about 100 parts by weight, in terms of platinum metal proper, per 1000 parts by weight of the total amount of compounds (A) and (B).

The polyglycerolated silicone elastomer of embodiments may be conveyed in the form of a gel in at least one hydrocarbon oil and/or one silicone oil. In such gels the polyglycerolated elastomer may be based on non-spherical particles.

In embodiments, the polyglycerolated silicone elastomers may be chosen from those sold under the names “KSG-710”, “KSG-810”, “KSG-820”, “KSG-830”, “KSG-840” by SHIN-ETSU.

The polyglycerolated silicone elastomer may be present in the exemplary embodiments of compositions in an amount ranging from about 0.1% to about 50% by weight, relative to the total weight of the composition, such as from about 0.1% to about 40% by weight, from about 0.5% to about 30% by weight, from about 0.5% to about 20% by weight, or from about 1% to about 10% by weight.

The silicone elastomer that can be used in the cosmetic compositions in accordance with embodiments may also be chosen from emulsifying silicone elastomers.

The silicone elastomer that can be used in the cosmetic compositions in accordance with embodiments may also be chosen from non-emulsifying silicone elastomers.

As used herein, the term “non-emulsifying” defines organopolysiloxane elastomers that do not contain a hydrophilic chain, such as polyoxyalkylenated or polyglycerolated units.

The spherical non-emulsifying silicone elastomer of embodiments may be chosen from elastomeric crosslinked organopolysiloxanes that may be obtained by (1) crosslinking addition reaction of a diorganopolysiloxane containing at least one hydrogen bonded to the silicon and of a diorganopolysiloxane having ethylenically unsaturated groups bonded to the silicon, optionally in the presence of a platinum catalyst; (2) dehydrogenation crosslinking condensation reaction between a diorganopolysiloxane containing hydroxyl end groups and a diorganopolysiloxane containing at least one hydrogen bonded to the silicon, optionally in the presence of an organotin compound; (3) crosslinking condensation reaction of a diorganopolysiloxane containing hydroxyl end groups and of a hydrolysable organopolysiloxane; (4) thermal crosslinking of an organopolysiloxane, optionally in the presence of an organic peroxide catalyst; or (5) crosslinking of an organopolysiloxane by means of high-energy radiation, such as gamma rays, ultraviolet rays or electron beams.

The elastomeric crosslinked organopolysiloxane of exemplary embodiments may be obtained by crosslinking addition reaction (A2) of a diorganopolysiloxane containing at least two hydrogens each bonded to a silicon, and (B2) of a diorganopolysiloxane having at least two ethylenically unsaturated groups bonded to the silicon, optionally in the presence (C2) of a platinum catalyst, as described in EP-A-295886.

In particular embodiments, the organopolysiloxane may be obtained by reacting a dimethylpolysiloxane containing dimethylvinylsiloxy end groups and a methylhydropolysiloxane containing trimethylsiloxy end groups, in the presence of a platinum catalyst.

The compound (A2) is the base reactant for the formation of elastomeric organopolysiloxanes of embodiments, and crosslinking is effected by an addition reaction of the compound (A2) with the compound (B2) in the presence of the catalyst (C2).

The compound (A2) may be chosen, in embodiments, from diorganopoly-siloxanes having at least two lower alkenyl groups (C₂-C₄, for example); the lower alkenyl group may be chosen from vinyl, allyl and propenyl groups. These lower alkenyl groups may be situated in any position of the organopolysiloxane molecule, but, in some embodiments, the lower alkenyl groups may be situated at the ends of the organopolysiloxane molecule. In embodiments, the organopolysiloxane (A2) may have a branched-chain, linear-chain, cyclic or network structure; and in particular embodiments, the organopolysiloxane has the linear-chain structure. The compound (A2) may have a viscosity ranging from the liquid state to the gum state, in embodiments, such as a viscosity of at least 100 centistokes at 25° C.

The organopolysiloxanes (A2) of embodiments may be chosen from methylvinylsiloxanes, methylvinylsiloxane-dimethylsiloxane copolymers, dimethylpolysiloxanes containing dimethylvinylsiloxy end groups, dimethylsiloxane-methylphenylsiloxane copolymers containing dimethylvinylsiloxy end groups, dimethylsiloxane-diphenylsiloxane-methylvinylsiloxane copolymers containing dimethylvinylsiloxy end groups, dimethylsiloxane-methylvinylsiloxane copolymers containing trimethylsiloxy end groups, dimethylsiloxane-methylphenylsiloxane-methylvinylsiloxane copolymers containing trimethylsiloxy end groups, methyl(3,3,3-trifluoropropyl)polysiloxanes containing dimethylvinylsiloxy end groups, and dimethylsiloxane-methyl(3,3,3-trifluoropropyl)siloxane copolymers containing dimethylvinylsiloxy end groups.

The compound (B2) may be, in embodiments, an organopolysiloxane having at least two hydrogens bonded to the silicon in each molecule, and may act as the crosslinker of the compound (A2).

In exemplary embodiments, the sum of the number of ethylenic groups per molecule of the compound (A2) and the number of hydrogen atoms bonded to the silicon per molecule of the compound (B2) is at least 4.

The compound (B2) may be in any molecular structure, and in particular embodiments has a linear-chain, branched-chain or cyclic structure.

The compound (B2) may have a viscosity at 25° C. ranging from about 1 to about 50 000 centistokes, in embodiments, so as to be well miscible with the compound (A).

In embodiments, compound (B2) may be added in an amount such that the molecular ratio between the total amount of hydrogen atoms bonded to the silicon in the compound (B2) and the total amount of all of the ethylenically unsaturated groups in the compound (A2) is in the range from about 1/1 to about 20/1.

The compound (B2) of embodiments may be chosen from methylhydropolysiloxanes containing trimethylsiloxy end groups, dimethylsiloxane-methylhydrosiloxane copolymers containing trimethylsiloxy end groups, and cyclic dimethylsiloxane-methylhydrosiloxane copolymers.

The compound (C2) is, in embodiments, the catalyst of the crosslinking reaction, and may be chosen from chloroplatinic acid, chloroplatinic acid-olefin complexes, chloroplatinic acid-alkenylsiloxane complexes, chloroplatinic acid-diketone complexes, platinum black and platinum on a support.

The catalyst (C2) may be added at from about 0.1 to about 1000 parts by weight, such as about 1 to about 100 parts by weight, in terms of platinum metal proper, per 1000 parts by weight of the total amount of the compounds (A2) and (B2).

In exemplary embodiments, additional organic groups may be bonded to the silicon in the organopolysiloxanes (A2) and (B2) described above, and include, for example, alkyl groups such as methyl, ethyl, propyl, butyl and octyl; substituted alkyl groups such as 2-phenylethyl, 2-phenylpropyl and 3,3,3-trifluoropropyl; aryl groups such as phenyl, tolyl and xylyl; substituted aryl groups such as phenylethyl; and substituted monovalent hydrocarbon groups such as an epoxy group, a carboxylate ester group or a mercapto group.

The elastomeric crosslinked organopolysiloxane particles of embodiments may be conveyed in the form of a gel composed of an elastomeric organopolysiloxane included in at least one hydrocarbon oil and/or silicone oil. Within these gels, the organopolysiloxane particles may be non-spherical particles.

The elastomeric crosslinked organopolysiloxane particles of embodiments may also be in powder form, such as in the form of a spherical powder.

Spherical non-emulsifying elastomers are described in JP-A-61-194009, EP-A-242219, EP-A-285886, EP-A-765656, the entire content of which is incorporated herein by reference.

Spherical non-emulsifying elastomers of embodiments may include those sold under the names “DC 9040”, “DC 9041”, “DC 9509”, “DC 9505” and “DC 9506” by DOW CORNING.

In embodiments, the spherical non-emulsifying silicone elastomer may also be present in the form of an elastomeric crosslinked organopolysiloxane powder coated with silicone resin, such as with silsesquioxane resin, as described for example in U.S. Pat. No. 5,538,793, the entire content of which is incorporated herein by reference. Elastomers of this kind are sold under the names “KSP-100”, “KSP-101”, “KSP-102”, “KSP-103”, “KSP-104” and “KSP-105” by SHIN-ETSU.

Other elastomeric crosslinked organopolysiloxanes in the form of spherical powders that may be included in embodiments may be hybrid silicone powders functionalized with fluoroalkyl groups, such as those sold under the name “KSP-200” by Shin Etsu; and hybrid silicone powders functionalized with phenyl groups, such as those sold under the name “KSP-300” by Shin Etsu.

In embodiments, the non-emulsifying spherical silicone elastomer may be present in the composition in an amount ranging from about 0.1% to about 95% by weight, relative to the total weight of the composition, such as from about 0.5% to about 75% by weight, from about 1% to about 50% by weight, from about 1% to about 40% by weight, or from about 1% to about 30% by weight.

According to particular embodiments, the compositions may comprise at least one fluid silicone compound.

According to particular embodiments, the compositions may comprise a non-volatile fluid silicone compound.

As used herein, a fluid compound encompasses any compound whose viscosity can be measured by any method known to the skilled person, such as the method described below or the ASTM-D-445 method.

The dynamic viscosity of the cosmetic compositions of embodiments at 25° C. may be measured by means of a rotational viscometer of Mettler RM 180 type. The dynamic viscosity of embodiments of cosmetic compositions may be in a range of from about 0.1 to about 120 Pa·s.

The Mettler RM 180 instrument (Rheomat) may be fitted with different spindles depending on the size order of the viscosity to be measured. For a viscosity varying from 0.18 to 4.02 Pa·s, the instrument is fitted with a spindle 3. For a viscosity varying from 1 to 24 Pa·s, the instrument is fitted with a spindle 4, and for a viscosity varying from 8 to 122 Pa·s the instrument is fitted with a spindle 5.

The viscosity is read off on the instrument in deviation units (DU). Tables supplied with the measuring instrument then allow the corresponding value in poises to be obtained.

The rotary speed of the spindle is approximately 200 rpm.

When the spindle is set in rotation, at a constant driven rotational speed (in this case 200 revolutions/minute), the viscosity value of the composition may vary over time until it reaches a constant value. Measurements are taken at regular intervals until constant measurement values are obtained. The viscosity value that has become constant over time is the value taken as being the dynamic viscosity value of the composition of embodiments. According to the 75 measurement system, the viscosity measurement is taken after 10 minutes.

The viscosity of exemplary embodiments of compositions may range from about 0.5 to about 50 Pa·s, such as from about 3.5 to about 25 Pa·s.

The addition of gelling agent is one of the formulating elements known to the skilled person for the purpose of modifying the viscosity of a composition.

Colorants

The cosmetic compositions of exemplary embodiments may incorporate one or more coloring agents, such as at least one colorant, organic or inorganic, which may be chosen from pigments and/or nacres conventionally used in cosmetic compositions.

As used herein, pigments encompass white or colored, mineral or organic particles that are insoluble in an aqueous solution and that color or opacify the resulting film.

The pigments of embodiments may be present in a proportion of from about 0.01% to about 15% by weight, such as from about 0.01% to about 10% by weight or from about 0.02% to about 5% by weight, relative to the total weight of the cosmetic composition. In embodiments, the pigments may be chosen from mineral pigments, such as titanium oxide, zirconium oxide or cerium oxide and also zinc oxide, iron oxide or chromium oxide, ferric blue, manganese violet, ultramarine blue and chromium hydrate.

The pigment of embodiments may be, for example, of sericite/brown iron oxide/titanium dioxide/silica structural type. Such pigments are sold for example under reference COVERLEAF NS or JS by CHEMICALS AND CATALYSTS, which has a contrast ratio of around 30.

The colorant of embodiments may also include a pigment having a structure, for example, of the type of silica microspheres containing iron oxide. An example of a pigment having this structure is that sold by MIYOSHI under reference PC BALL PC-LL-100 P, which is composed of silica microspheres containing yellow iron oxide.

The colorants of embodiments may be chosen from organic pigments, such as carbon black, D & C pigments, lakes based on cochineal carmine, on barium, strontium, calcium or aluminum, or else the diketopyrrolopyrroles (DPP) described in EP-A-542669, EP-A-787730, EP-A-787731, and WO-A-96/08537.

As used herein, “nacres” encompass colored particles of any shape, iridescent or non-iridescent, that are produced, for example, by certain mollusks in their shell or are synthesized, and that exhibit a color effect by optical interference.

The nacres of embodiments may be chosen from nacreous pigments such as titanium mica coated with an iron oxide, mica coated with bismuth oxichloride, titanium mica coated with chromium oxide, titanium mica coated with an organic dye, and nacreous pigments based on bismuth oxichloride. The nacreous pigment of embodiments may also include mica particles having at least two successive layers of metal oxides and/or of organic colorants superposed on the mica surface.

Mention may also be made, as examples of nacres for use in embodiments, of natural mica coated with titanium dioxide, with iron oxide, with natural pigment or with bismuth oxichloride.

Among nacres available on the market mention are TIMICA, FLAMENCO and DUOCHROME (based on Mica), which are sold by ENGELHARD, the TIMIRON nacres sold by MERCK, the PRESTIGE mica-based nacres sold by ECKART, and the synthetic-mica-based SUNSHINE nacres sold by SUN CHEMICAL.

The nacres of exemplary embodiments may possess a yellow, pink, red, bronze, orange, brown, gold and/or copper color or glint.

By way of illustration, nacres that may be used in embodiments include, for example, the golden nacres sold by ENGELHARD under the name Brilliant gold 212G (TIMICA), Gold 222C (Cloisonne), Sparkle gold (TIMICA), Gold 4504 (Chromalite) and Monarch gold 233X (Cloisonne); the bronze nacres sold by MERCK under the name Bronze fine (17384) (Colorona) and Bronze (17353) (Colorona) and by ENGELHARD under the name Super bronze (Cloisonne); the orange nacres sold by ENGELHARD under the name Orange 363C (Cloisonne) and Orange MCR¹⁰¹ (Cosmica) and by MERCK under the name Passion orange (Colorona) and Matt orange (17449) (Microna); the brown-hued nacres sold by ENGELHARD under the name Nu-antique copper 340XB (Cloisonne) and Brown CL4509 (Chromalite); the copper-glint nacres sold by ENGELHARD under the name Copper 340A (TIMICA); the red-glint nacres sold by MERCK under the name Sienna fine (17386) (Colorona); the yellow-glint nacres sold by ENGELHARD under the name Yellow (4502) (Chromalite); the gold-glint red-hued nacres sold by ENGELHARD under the name Sunstone G012 (Gemtone); the pink nacres sold by ENGELHARD under the name Tan opal G005 (Gemtone); the gold-glint black nacres sold by ENGELHARD under the name Nu antique bronze 240 AB (TIMICA), the blue nacres sold by MERCK under the name Matt blue (17433) (Microna), the silver-glint white nacres sold by MERCK under the name XIRONA Silver, and the green-golden pinkish orangish nacres sold by MERCK under the name Indian summer (XIRONA), and mixtures thereof.

The cosmetic compositions of embodiments may also include water-soluble or fat-soluble dyes in an amount ranging from 0.01% to 10% by weight, such as from 0.01% to 5% by weight, relative to the total weight of the cosmetic composition. The fat-soluble dyes for use in embodiments may include, for example, Sudan Red, DC Red 17, DC Green 6, β-carotene, soya oil, Sudan Brown, DC Yellow 11, DC Violet 2, DC orange 5, and quinoline yellow. The water-soluble dyes of embodiments may include, for example, beetroot juice and methylene blue.

The cosmetic compositions of embodiments may also include at least one material having a specific optical effect.

The specific optical effect of embodiments is different from a simple, conventional hue effect; the specific optical effect of embodiments is a unified and stabilized effect of the kind produced by conventional colorants such as monochromatic pigments, for example. As used herein, “stabilized” signifies absence of an effect of variability of color with the angle of observation or in response to a temperature change.

For example, the material having a specific optical effect in embodiments may be chosen from particles having a metallic glint, goniochromatic coloring agents, diffracting pigments, thermochromic agents, optical brighteners, and also fibers, which may be interference type fibers. Different materials having specific optical effects may be combined in such a way as to produce the simultaneous manifestation of two effects or the manifestation of another effect in embodiments.

The metallic-glint particles of embodiments may be chosen from:
particles of at least one metal and/or of at least one metal derivative;
particles including a single-substance or multi-substance, organic or inorganic substrate, at least partly coated with at least one metal-glint layer that includes at least one metal and/or at least one metal derivative, and
mixtures of said particles.

Among the metals that can be present in said particles mention may be made, for example, of Ag, Au, Cu, Al, Ni, Sn, Mg, Cr, Mo, Ti, Zr, Pt, Va, Rb, W, Zn, Ge, Te, Se and mixtures or alloys thereof. Ag, Au, Cu, Al, Zn, Ni, Mo, Cr and their mixtures or alloys (for example bronzes and brasses) may be sued as the metals in particular embodiments.

As used herein, “metal derivatives” encompass compounds derived from metals, especially oxides, fluorides, chlorides and sulphides.

Such particles may be, in exemplary embodiments, aluminum particles, such as those sold under the names STARBRITE 1200 EAC® by SILBERLINE, and METALURE® by ECKART.

Exemplary embodiments may include metallic powders of copper or of alloy mixtures such as references 2844 sold by RADIUM BRONZE; metal pigments such as aluminum or bronze, such as those sold under the names ROTOSAFE 700 from ECKART; the silica-sheathed aluminum particles sold under the name VISIONAIRE BRIGHT SILVER from ECKART; and the metal alloy particles such as silica-sheathed bronze (copper and zinc alloy) powders sold under the name VISIONAIRE BRIGHT NATURAL gold from ECKART.

The particles of embodiments may be particles including a glass substrate, such as those sold by NIPPON SHEET GLASS under the names MICROGLASS METASHINE.

The goniochromatic colouring agent may be chosen in embodiments from, for example, multilayer interference structures and liquid-crystal colouring agents.

In compositions according to embodiments, symmetrical multilayer interference structures may be used, including, for example, the following structures: Al/SiO₂/Al/SiO₂/Al, pigments having this structure being sold by the company DUPONT DE NEMOURS; Cr/MgF₂/Al/MgF₂/Cr, pigments having this structure being sold under the name CHROMAFLAIR by FLEX; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name SICOPEARL by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name XIRONA® by MERCK (Darmstadt). By way of example, pigments may be, in embodiments, the pigments of silica/titanium oxide/tin oxide structure sold under the name XIRONA MAGIC by MERCK, the pigments of silica/brown iron oxide structure sold under the name XIRONA INDIAN SUMMER by MERCK; the pigments of silica/titanium oxide/mica/tin oxide structure sold under the name XIRONA CARIBBEAN BLUE by MERCK, and the INFINITE COLORS pigments from the company SHISEIDO. Depending on the thickness and the nature of the various layers, different effects are obtained. Thus, with the Fe₂O₃/SiO₂/Al/SiO₂/Fe₂O₃ structure, the color changes from green-golden to red-grey for SiO₂ layers of 320 to 350 nm; from red to golden for SiO₂ layers of 380 to 400 nm; from violet to green for SiO₂ layers of 410 to 420 nm; from copper to red for SiO₂ layers of 430 to 440 nm.

Examples of pigments with a polymeric multilayer structure that may be used in embodiments include those sold by 3M under the name COLOR GLITTER.

Examples of liquid-crystal goniochromatic particles that may be used in embodiments include those sold by CHENIX and the products sold under the name HELICONE® HC by WACKER.

Fillers

In exemplary embodiments, the cosmetic compositions may also include at least one filler, of organic or mineral nature, which may allow improved stability with regard to exudation to be imparted.

As used herein, the term “filler” encompasses colorless or white, solid particles of any form, which are in an insoluble and dispersed form in the medium of the composition. Mineral or organic in nature, fillers may give body or rigidity to the composition, and/or softness, and/or a matte effect and/or uniformity to the composition.

The fillers used in the compositions of embodiments may be of lamellar, globular, spherical or fibrous form or in any other form intermediate between these defined forms.

The fillers according to embodiments may or may not be surface-coated; for example, fillers may be surface-treated with silicones, amino acids, fluoro derivatives or any other substance that promotes the dispersion and compatibility of the filler in the composition.

As used herein, the terms “mineral fillers” and “inorganic fillers” are interchangeable.

The mineral fillers that may be used in embodiments may be chosen from talc, mica, silica, trimethyl siloxysilicate, kaolin, bentone, precipitated calcium carbonate, magnesium carbonate, magnesium hydrocarbonate, hydroxyapatite, boron nitride, hollow silica microspheres (Silica Beads from Maprecos), glass or ceramic microcapsules, silica-based fillers, for instance AEROSIL 200 and AEROSIL 300; SUNSPHERE L-31 and SUNSPHERE H-31 sold by Asahi Glass; CHEMICELEN sold by Asahi Chemical; and composites of silica and of titanium dioxide, for instance the TSG series sold by NIPPON SHEET GLASS, and mixtures thereof.

The organic fillers that may be used in embodiments may be chosen from polyamide powder (NYLON® ORGASOL from Atochem); poly-b-alanine powder and polyethylene powder; polytetrafluoroethylene (TEFLON®) powders; lauroyllysine; starch; powders of tetrafluoroethylene polymers; hollow polymer microspheres such as EXPANCEL (NOBEL INDUSTRIE); precipitated calcium carbonate; magnesium carbonate; magnesium hydrocarbonate; metal soaps derived from organic carboxylic acids containing from 8 to 22 carbon atoms including those containing from 12 to 18 carbon atoms, for example zinc stearate, magnesium stearate or lithium stearate, zinc laurate or magnesium myristate, and POLYPORE® L 200 (Chemdal Corporation); silicone resin microbeads (for example TOSPEARL® from Toshiba); and polyurethane powders, such as powders of crosslinked polyurethane including a copolymer that includes trimethylol hexyllactone. In particular embodiments, the organic filler may be a hexamethylene diisocyanate/trimethylol hexyllactone polymer. Such particles are commercially available, for example, under the name PLASTIC POWDER D-400® or PLASTIC POWDER D-800® from the company TOSHIKI, and mixtures thereof.

The fillers of embodiments may be present in a proportion of from 0.001% to 35% or of from 0.5% to 15% of the total weight of the composition.

The filler of embodiments may be, for example, a filler with a mean particle size of less than 100 μm, such as between 1 and 50 μm or between 4 and 20 μm.

According to particular embodiments, the composition includes at least one filler present in a proportion of from 0.01% to 60% of the total weight of the composition, such as from 0.5% to 20% or from 1% to 10% by weight relative to the total weight of the composition.

Additives

The cosmetic compositions according to exemplary embodiments may also comprise any additive commonly used in the field, such as, for example, additives chosen from gelling agents as described in international patent application publication WO 2004/55080, the content of which is incorporated herein by reference, surfactants as described in application FR 2834452, the content of which is incorporated herein by reference, film formers as described in application FR 0450540, the content of which is incorporated herein by reference; and where appropriate, film-forming auxiliaries; antioxidants; essential oils; preservatives; perfumes; neutralizing agents; moisturizers; antiseptics; vitamins such as vitamin B3, vitamin E and derivatives thereof; and UV protectants.

According to particular embodiments, the silicone surfactants of non-crosslinked type are included in the compositions.

According to particular embodiments, the may be devoid of ammonium-type surfactants.

Of course, the person skilled in the art will take care to select the optional additive(s) added to the cosmetic composition according to embodiments in such a way that the intrinsic properties of the composition are not, or not substantially, adversely affected by the addition.

According to exemplary embodiments, the silicone polymer of general formula (I) may be chosen from polyglyceryl-3 polydimethylsiloxyethyl dimethicone, lauryl polyglyceryl-3 polydimethylsiloxyethyl dimethicone, polyglyceryl-3 disiloxane dimethicone, and mixtures thereof.

According to exemplary embodiments, the silicone polymer of general formula (I) may be chosen from the silicone polymers sold by SHIN-ETSU under references KF 6100®, KF 6104′, KF 6105®, and mixtures thereof.

According to exemplary embodiments, the cosmetic composition includes polyglyceryl-3 polydimethylsiloxyethyl dimethicone and, as film former, acrylate/stearyl acrylate/dimethicone methacrylate copolymer, such as that sold under reference KP 561® by SHIN-ETSU.

According to exemplary embodiments, the cosmetic composition includes polyglyceryl-3 polydimethylsiloxyethyl dimethicone and at least one wax, which may be chosen from polyethylene waxes, candelilla wax, hydrogenated cocoglyceride wax, and mixtures thereof.

According to exemplary embodiments, the cosmetic composition includes polyglyceryl-3 polydimethylsiloxyethyl dimethicone and at least one hydrocarbon ester containing less than 40 carbon atoms, which may be chosen from stearyl heptanoate, isononyl isononanoate and isotridecyl isononanoate, and mixtures thereof.

In some exemplary embodiments, individual components may belong at one or more different classes of compounds included in the embodiments. For example, isononyl isononanoate may be both a non-volatile oil and a hydrocarbon ester containing less than 40 carbon atoms.

Accordingly, it is not outside the bounds of customary work of the person skilled in the art to adjust the amount of a compound belonging to more than one different class of product to obtain a desired effect.

The cosmetic composition according to exemplary embodiments may be in the form of a lip makeup product, such as a lipstick, or a lip balm.

The examples of compositions below are given by way of illustration and are not to be construed as in any way limiting.

### EXAMPLE 1

### Lipstick

A lipstick is prepared that includes the components set forth in Table I.

TABLE I

| | Percentages |
|---|---|
| | by mass |
| Hydrogenated polyisobutene (Parleam HV from NOF) | 4 |
| Isononyl isononanoate | 12 |
| 2-Octyldodecanol | 4.5 |
| Diisostearyl malate | 33.9 |
| Triglycerides of lauric/palmitic/cetyl/stearic (50/20/10/10) | 4 |
| acids (Softisan 100 ® from Sasol) | |
| N-lauroyl L-lysine | 1 |
| Acrylates/stearyl acrylate/dimethicone methacrylate | 4 |
| copolymer (KP 561 P ® from Shin Etsu) | |
| Dimethicone 6 cSt (KF 96) | 4 |
| Polyglyceryl-3 polydimethylsiloxyethyl dimethicone | 13 |
| (KF6104 ® from Shin Etsu) | |
| Preservative | qs |
| Polyethylene wax (MW 500) | 6.6 |
| Polyethylene wax (MW 400) | 3.8 |
| Hydrophobic pyrogenic silica surface-treated with | 2 |
| dimethylsilane (Aerosil R 972 ® from Degussa) | |
| Pigments | 7.0 |
| Simethicone (Antifoam C ® from Dow Corning) | 0.2 |
| Total | 100 |

Procedure

An oily phase is prepared by mixing, with heating to approximately 95° C., isononyl isononanoate, 2-octyldodecanol and diisostearyl malate with polyglyceryl-3 polydimethylsiloxyethyl dimethicone and dimethicone oil. The oily phase thus prepared is stirred at approximately 95° C. and fillers (N-lauroyl-L-lysine and pyrogenic silica) are added to the mixture.

The waxes, the pigments in the form of a pigment paste, the hydrogenated polyisobutene and the simethicone are then added to the mixture.

The mixture thus obtained is subsequently poured into a lipstick mold and left to cool until a solid composition is obtained.

Thereafter, the staying power, comfort and gloss of this composition are measured in accordance with the protocols described above.

Also measured, using the above-described methods, are the staying power and gloss of two commercial products:
Control A: JELLY PLUMPY sold by Maybelline, containing phenyltrimethicone as silicone polymer;
Control B: AUBE ROUGE GLACÉ sold by Kao and containing the glycerolated silicone of formula below as silicone polymer

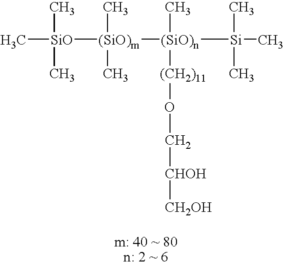

The results obtained are reported in Table II below.

TABLE II

| | Water | Oil | | Comfort | Average |
|---|---|---|---|---|---|
| Formula | resistance | resistance | Transfer | index | gloss |
| Control A | 14.4 | 23.4 | 47.3 | | 42.5 |
| Control B | 7.0 | 8.5 | 42.0 | 85.6 | 48.2 |
| Invention | 5.9 | 7.9 | 32.1 | 99.5 | 48.3 |

In these measurements, the color of the inner surface of the forearm is such that L*=63.9, a*=8.4, b*=13.3, and the color of the paper tissue is such that L*=97.9, a*=0.6 and b*=3.3.

The lipstick of Example 1 has a better staying power and better comfort than the available commercial products tested as Control A and Control B, for an equivalent or equal gloss.

Moreover, the lipstick of Example 1 migrates three to four times less than the Control B.

### EXAMPLES 2 AND 3

Lipsticks

Lipsticks are prepared including the components set forth in Table III. The percentages are by weight.

TABLE III

| | Example 2 | Example 3 |
|---|---|---|
| Hydrogenated polyisobutene (PARLEAM | | 4 |
| HV from NOF) | | |
| Hydrogenated polyisobutene (PARLEAM | 6 | |
| LITE from NOF) | | |
| Isononyl isononanoate | 8 | 12 |
| 2-Octyldodecanol | 4.5 | 4.5 |
| Diisostearyl malate | 33.9 | 23.9 |
| Polyglyceryl-2 diisostearate (COSMOL | | 5 |
| 42 V from Nishin Oil) | | |
| Polyglyceryl-2 triisostearate (COSMOL | | 4 |
| 43 N from Nishin Oil) | | |
| Sorbitan Sesquioleate (COSMOL | | 1 |
| 82 from Nishin Oil) | | |
| Triglycerides of lauric/palmitic/ | 3 | 4 |
| cetyl/stearic (50/20/10/10) | | |
| acids (SOFTISAN 100 ® from Sasol) | | |
| N-lauroyl-L-lysine | 1 | 1 |
| Polyvinyl laurate | 3 | |
| Acrylates/stearyl acrylate/dimethicone | 2 | 4 |
| methacrylate copolymer (KP 561 | | |
| P ® from Shin Etsu) | | |
| Dimethicone 6 cSt (KF 96) | 5 | 4 |
| Polyglyceryl-3 polydimethylsiloxyethyl | 13 | 13 |
| dimethixone (KF6104 ® from Shin Etsu) | | |
| Preservative | qs | |
| Polyethylene wax (MW 500) | 5.5 | 6.6 |
| Polyethylene wax (MW 400) | 1.1 | |
| Candelilla wax | 4.8 | |
| Microcrystalline wax | | 3.8 |
| Hydrophobic pyrogenic silica surface- | 2 | 2 |
| treated with dimethylsilane (AEROSIL | | |
| R 972 ® from Degussa) | | |
| Pigments | 7.0 | 7 |
| Simeethicone (ANTIFOAM C ® from | 0.2 | 0.2 |
| DOW CORNING) | | |
| Total | 100 | 100 |

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention.

What is claimed is:

1. A cosmetic composition comprising, in a physiologically acceptable medium, at least one hydrocarbon ester comprising less than 40 carbon atoms and at least one silicone polymer of general formula (I):
R¹ₐR²_{b}R³_{c}SiO_{(4-a-b-c)/2} (I)
in which:
a is in a range of from about 1 to about 2.5,
b and c each are independently in a range of from about 0.001 to about 1.5,
R¹, which may be identical or different at each occurrence, is chosen from:
C₁ to C₃₀ alkyl radicals,
C₁ to C₃₀ alkyl radicals, substituted by one or more fluorine atoms, amino and/or carboxyl groups,
aryl and aralkyl radicals,
radicals of general formula (II):
C_{d}H_{2d}—O—(C₂H₄O)ₑ(C₃H₆O)_{f}R⁴ (II)
wherein:
R⁴ is chosen from C₁ to C₃₀ hydrocarbon radicals and radicals R⁵—(CO)—, in which R⁵ is chosen from C₁ to C₃₀ hydrocarbon radicals,
d is an integer in a range of from 0 to about 15, and
e and f are each integers that are independently in a range of from 0 to about 50,
and combinations thereof,
R² is a radical represented by the general formula (III):
-Q-O—X (III)
wherein:
Q is chosen from divalent C₂ to C₂₀ hydrocarbon radicals that can include at least one ether bond and/or at least one ester bond, and
X is chosen from polyhydroxylated hydrocarbon radicals,
wherein when R² is a radical of general formula (IIIA):
—C₃H₆—O[CH₂CH(OH)CH₂O]ₙH (IIIA)
in which n is an integer in a range of from 1 to 5, R¹ is not a C₁₋₂ alkyl radical,
and R³ is an organosiloxane group of general formula (IV): 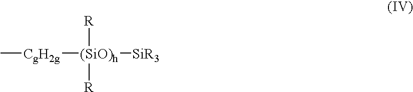
wherein:
the radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500,
and wherein the composition comprises less than about 15% by weight of volatile oil relative to a total weight of the composition.

2. A cosmetic composition comprising, in a physiologically acceptable medium, at least one hydrocarbon monoester comprising less than 40 carbon atoms and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
C_{d}H_{2d}—O—(C₂H₄O)ₑ(C₃H₆O)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50,
and combinations thereof,
R² is a radical represented by the general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
wherein when R² is a radical of general formula (IIIA):
—C₃H₆—O[CH₂CH(OH)CH₂O]ₙH (IIIA)
in which n is an integer in a range of from 1 to 5, R¹ is not a C₁₋₂ alkyl radical,
and R³ is an organosiloxane of general formula (IV): 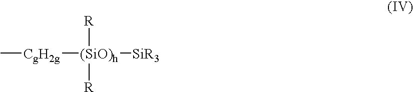
wherein:
radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500.

3. An anhydrous cosmetic composition comprising, in a physiologically acceptable medium, at least one hydrocarbon ester comprising less than 40 carbon atoms and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50,
and combinations thereof,
R² is a radical of general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
wherein when R² is a radical of general formula (IIIA):
—C₃H₆—O[CH₂CH(OH)CH₂O]ₙH (IIIA)
in which n is an integer in a range of from 1 to about 5, R¹ is not a C₁₂ alkyl radical,
R³ is an organosiloxane of general formula (IV): 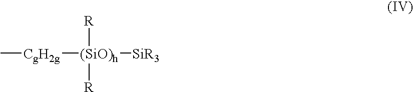
wherein:
the radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500.

4. An anhydrous cosmetic composition comprising, in a physiologically acceptable medium, at least one fluid silicon compound and at least one hydrocarbon ester comprising less than 40 carbon atoms and/or at least one volatile oil, and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50,
and combinations thereof,
R² is a radical of general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
and R³ is an organosiloxane of general formula (IV): 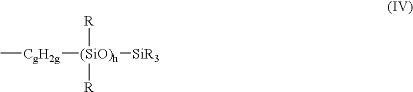
wherein:
the radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500.

5. An anhydrous cosmetic composition comprising, in a physiologically acceptable medium, at least one hydrocarbon ester containing less than 40 carbon atoms, at least one volatile oil and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆₀)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50,
and combinations thereof,
R² being a radical of general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
and R³ being an organosiloxane of general formula (IV): 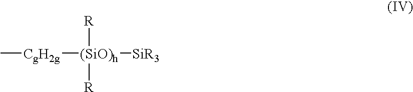
wherein:
the radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500.

6. An anhydrous cosmetic composition comprising, in a physiologically acceptable medium, at least two esters containing less than 40 carbon atoms and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50,
and combinations thereof,
R² being a radical of general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
and R³ being an organosiloxane of general formula (IV): 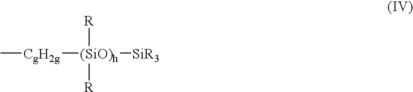
wherein:
radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500.

7. An anhydrous cosmetic composition comprising, in a physiologically acceptable medium, at least one hydrocarbon ester containing fewer than 22 carbon atoms and at least one silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals, and
radicals of general formula (II):
—C_{d}H_{2d}—O—(C₂H₄O)ₑ(C₃H₆₀)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50,
and combinations thereof,
R² being a radical of general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
and R³ being an organosiloxane of general formula (IV): 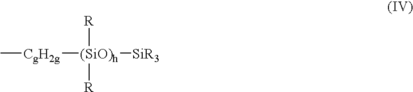
wherein:
radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500.

8. The cosmetic composition according to claim 1, wherein the silicone polymer is a compound of general formula (I) in which R¹ is a C₁ to C₁₀.

9. The cosmetic composition according to claim 1, wherein the silicone polymer is a compound of general formula (I) in which:
a is in a range of from about 1 to about 1.4,
b and c are each independently in a range of from about 0.02 to about 0.04,
R¹ is a C₁ to C₁₀ alkyl radical,
R² is represented by the formula (IIIA):
—C₃H₆O[CH₂CH(OH)CH₂O]ₙH (IIIA)
in which n is in a range of from about 1 to about 5,
and R³ is represented by the formula (IVA):
—C₂H₄(CH₃)₂SiO[(CH₃)₂SiO]ₘSi(CH₃)₃ (IVA)
in which m is in a range of from about 3 to about 9.

10. The cosmetic composition according to claim 9, wherein the silicone polymer is a compound of general formula (I):
R¹ₐR²_{b}R^{a}_{c}SiO_{(4-a-b-c)/2} (I)
in which:
a is in a range of from about 1 to about 1.4,
b and c are each independently in a range of from about 0.02 to about 0.04,
R¹ is a methyl radical,
R² is represented by the formula (IIIA) in which n is in a range from about 1 to about 5, and
R³ is represented by the formula (IVA) in which m is in a range from about 3 to about 9.

11. The cosmetic composition according to claim 2, further comprising at least one volatile oil.

12. The cosmetic composition according to claim 4, wherein said volatile oil is present at less than 15% by weight relative to a total weight of the composition.

13. The cosmetic composition according to claim 1, wherein said volatile oil is chosen from:
hydrocarbon oils having from 8 to 16 carbon atoms,
linear or cyclic silicone oils,
fluorinated solvents, and
mixtures thereof.

14. The cosmetic composition according to claim 1, wherein said hydrocarbon ester comprises a monoester.

15. The cosmetic composition according to claim 2, wherein said monoester corresponds to the formula RCOOR′ in which RCOO represents a fatty acid residue containing 2 to 28 carbon atoms and R′ represents a hydrocarbon chain containing from 1 to 28 carbon atoms, and wherein said monoester contains at least 10 carbon atoms.

16. The cosmetic composition according to claim 1, wherein said hydrocarbon ester contains less than 22 carbon atoms and is not a volatile oil.

17. The cosmetic composition according to claim 1, wherein said hydrocarbon ester is chosen from synthetic esters; hydroxylated esters; fatty alcohol heptanoates, octanoates and decanoates; esters of neopentanoic acid; esters of isononanoic acid; esters of isopropyl alcohol; alkyl benzoate; polyethylene glycol diheptanoate; propylene glycol di-2-ethylhexanoate; and mixtures thereof.

18. The cosmetic composition according to claim 1, further comprising at least one silicone compound chosen from silicone oils, silicone gums, silicone resins and silicone elastomers.

19. The cosmetic composition according to claim 18, wherein said silicone compound comprises a silicone resin or an acrylate/stearyl acrylate/silicone methacrylate copolymer.

20. The cosmetic composition according to claim 18, wherein said silicone compound is fluid.

21. The cosmetic composition according to claim 1, further comprising at least one non-volatile oil chosen from oils of animal, vegetable, mineral and synthetic origin, and mixtures thereof.

22. The cosmetic composition according to claim 21, wherein said non-volatile oil is chosen from hydrogenated polyisobutene, dipentaerythritol tetrahydroxystearate/tetraisostearate, 2-octyldodecanol, diisostearyl malate, and mixtures thereof.

23. The cosmetic composition according to claim 1, further comprising at least one silicone oil.

24. The cosmetic composition according to claim 23, wherein said silicone oil is chosen from:
linear or cyclic silicone oils,
silicone oils, and
mixtures thereof.

25. The cosmetic composition according to claim 1, wherein the cosmetic composition has a transfer value not greater than 45.

26. The cosmetic composition according to claim 1, wherein the cosmetic composition has a color fastness to water not greater than 15.

27. The cosmetic composition according to claim 1, wherein the cosmetic composition has a color fastness to oil not greater than 25.

28. The cosmetic composition according to claim 1, wherein the cosmetic composition has an average gloss of at least 30.

29. The cosmetic composition according to claim 1, wherein the cosmetic composition is in cast form.

30. The cosmetic composition according to claim 1, further comprising at least one colorant.

31. The cosmetic composition according to claim 1, further comprising at least one compound chosen from waxes, pasty fatty substances, and mixtures thereof.

32. The cosmetic composition according to claim 31, wherein the wax is chosen from waxes of animal, vegetable and synthetic origin, and mixtures thereof.

33. The cosmetic composition according to claim 1, further comprising at least one filler.

34. The cosmetic composition according to claim 1, wherein the cosmetic composition comprises at least one of a skin care composition, a lip care composition and a makeup composition.

35. A method of preparing the cosmetic composition according to claim 4, comprising:
combining at least one hydrocarbon ester comprising less than 40 carbon atoms, at least one silicone compound and/or at least one volatile oil different from the silicone polymer of general formula (I):
R¹ₐR²_{b}R³_{c}SiO_{(4-a-b-c)/2} (I)
in which:
a is in a range of from about 1 to about 2.5,
b and c are each independently in a range of from about 0.001 to about 1.5,
R¹, which may be identical or different at each occurrence, is a radical chosen from the group consisting of:
C₁ to C₃₀ alkyl radicals,
C₁ to C₃₀ alkyl radicals substituted with one or more fluorine atoms, amino and/or carboxyl groups,
aryl and aralkyl radicals,
radicals of general formula (II):
—C_{d}H_{2d}—O—(C₂H₄O)ₑ(C₃H₆O)_{f}R⁴ (II)
wherein:
R⁴ is a radical chosen from the group consisting of C₁ to C₃₀ hydrocarbon radicals and R⁵—(CO)— radicals in which R⁵ is a radical chosen from the group consisting of C₁ to C₃₀ hydrocarbon radicals,
d is an integer in a range of from 0 to about 15, and
e and f are each independently integers in a range of from 0 to about 50,
and combinations thereof,
R² is a radical of general formula (III):
-Q-O—X (III)
wherein:
Q is a divalent C₂ to C₂₀ hydrocarbon radical that can include at least one ether bond and/or at least one ester bond, and
X is a polyhydroxylated hydrocarbon radical,
R³ is an organosiloxane of general formula (IV): 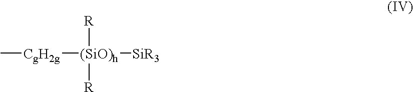
wherein:
radicals R are each radicals independently chosen from the group consisting of C₁ to C₃₀ alkyl radicals, C₁ to C₃₀ alkyl radicals substituted with one or more fluorine atoms, and aryl and aralkyl radicals,
g is an integer in a range of from 1 to about 5, and
h is an integer in a range of from 0 to about 500.

36. The method according to claim 35, wherein said hydrocarbon ester said hydrocarbon ester comprising less than 40 carbon atoms is a monoester.

37. The method according to claim 35, wherein said silicone compound is chosen from silicone oils, silicone gums, silicone resins and silicone elastomers.

38. The method according to claim 35, wherein said volatile oil is chosen from:
hydrocarbon oils having from 8 to 16 carbon atoms,
linear or cyclic silicone oils, and
fluorinated solvents, and
mixtures thereof.

39. The method according to claim 35, wherein said cosmetic composition comprises less than 15% by weight of volatile oil relative to a total weight of the composition.

40. The method according to claim 35, wherein said cosmetic composition has a transfer value not greater than 45.

41. The method according to claim 25, wherein said composition has a color fastness to water not greater than 15.

42. The method according to claim 35, wherein said composition has a colour fastness to oil not greater than 25.

43. The method according to claim 35, wherein said composition has an average gloss of at least 30.

44. The method according to claim 35, wherein said cosmetic composition is anhydrous.

45. A method of making up and/or caring for keratin materials comprising:
applying the cosmetic composition of claim 1 to one or more keratin materials chosen from the group consisting of skin, mucosae, nails, eyelashes and hair.
